(19) United States
(12) Patent Application Publication
(10) Pub. No.: US 2019/0317608 A1
(43) Pub. Date: Oct. 17, 2019

(54) TRANSMISSIVE HEAD-MOUNTED DISPLAY APPARATUS, DISPLAY CONTROL METHOD, AND COMPUTER PROGRAM

(71) Applicant: SEIKO EPSON CORPORATION, Tokyo (JP)

(72) Inventors: Takehiro ONO, Chino-shi (JP); Masahide TAKANO, Matsumoto-shi (JP)

(73) Assignee: SEIKO EPSON CORPORATION, Tokyo (JP)

(21) Appl. No.: 16/382,559

(22) Filed: Apr. 12, 2019

(30) Foreign Application Priority Data
Apr. 13, 2018 (JP) 2018-077562

(51) Int. Cl. G06F 3/01; G06K 9/00; A61B 5/0488; A61B 5/11; A61B 5/04

(52) U.S. Cl. CPC G06F 3/017; G06F 3/011; G06K 9/00355; A61B 2503/12; A61B 5/1123; A61B 5/04012; A61B 5/0488

(57) ABSTRACT

A transmissive head-mounted display apparatus includes an image display unit configured to transmit an external scenery and display an image of a display target viewable with the external scenery, a kinetic information acquisition unit configured to acquire information about a muscle movement, detected by a muscle activity detection device configured to detect a muscle movement of a body of a user of the transmissive head-mounted display apparatus, a hand gesture estimation unit configured to estimate a hand gesture specified beforehand, based on the muscle movement indicated in the information that has been acquired, and an input signal generation unit configured to generate an input signal specified beforehand in accordance with the hand gesture that is estimated.

The present application is based on, and claims priority from JP Application Serial Number 2018-077562, filed Apr. 13, 2018, the disclosure of which is hereby incorporated by reference herein in its entirety.

### BACKGROUND

### 1. Technical Field

The present disclosure relates to a transmissive head-mounted display apparatus.

### 2. Related Art

As a head-mounted display apparatus (Head-Mounted Display (HMD)) mounted on a user's head to display images and the like within the user's visual field, a transmissive head-mounted display apparatus is known that transmits an external scenery viewable with the display images. Another controller than a main device is used as an input device for controlling the transmissive head-mounted display apparatus. The controller includes a plurality of operating units such as a software keyboard to be displayed on a liquid crystal display, and buttons and a track pad. Various techniques capable of detecting a movement of an instructing body such as a thumb or a finger of a hand and executing processing in accordance with the movement of the instructing body have been proposed. For example, JP-A-8-115408 discloses a sign language recognition device configured to convert a movement for sign language into an electric signal, convert the electric signal into a speech language, and output the speech language.

The inventors of the present disclosure have found that, when a movement of an instructing body, as described in JP-A-8-115408, and a movement (gesture) of a body of a user are used as operations for a transmissive head-mounted display apparatus, and when such a movement is performed outside an imaging range of a camera provided in a main device, the movement cannot be detected or precision in detection could lower.

### SUMMARY

An exemplary embodiment of the present disclosure provides a transmissive head-mounted display apparatus. The transmissive head-mounted display apparatus includes an image display unit configured to transmit an external scenery and display an image of a display target viewable with the external scenery, a kinetic information acquisition unit configured to acquire information about a muscle movement, detected by a muscle activity detection device configured to detect a muscle movement of a body of a user of the transmissive head-mounted display apparatus, a hand gesture estimation unit configured to estimate a hand gesture specified beforehand, based on the muscle movement indicated in the information that has been acquired, and an input signal generating unit configured to generate an input signal specified beforehand in accordance with the hand gesture that is estimated.

### BRIEF DESCRIPTION OF THE DRAWINGS

FIG. 1 is an explanatory diagram illustrating a schematic configuration of a transmissive head-mounted display apparatus according to an exemplary embodiment of the present disclosure.

FIG. 2 is a plan view illustrating a configuration of a main part of an optical system included in an image display unit.

FIG. 3 is a diagram illustrating a configuration of a main part of the image display unit as viewed from a user.

FIG. 4 is a diagram illustrating an angle of view of a camera.

FIG. 5 is a functional block diagram illustrating a configuration of the HMD.

FIG. 6 is a functional block diagram illustrating a configuration of a control device.

FIG. 7 is an explanatory diagram illustrating an example of augmented reality display provided by the HMD.

FIG. 8 is an explanatory diagram schematically illustrating myoelectric sensors attached to hands and arms of a user.

FIG. 9 is a flowchart illustrating how character input processing proceeds.

FIG. 10 is an explanatory diagram schematically illustrating a hand gesture indicative of “start of character entry”.

FIG. 11 is an explanatory diagram schematically illustrating temporal changes in myoelectric potential when the hand gesture indicative of “start of character entry” is performed.

FIG. 12 is an explanatory diagram schematically illustrating the user's field of view after execution of step S120.

FIG. 13 is an explanatory diagram schematically illustrating the user's field of view after execution of step S150.

FIG. 14 is an explanatory diagram schematically illustrating a hand gesture indicative of “determination of input signal”.

FIG. 15 is an explanatory diagram schematically illustrating temporal changes in myoelectric potential when the hand gesture indicative of “determination of input signal” is performed.

### DESCRIPTION OF EXEMPLARY EMBODIMENTS

### A. Exemplary Embodiment

A1. Overall Configuration of Transmissive Head-Mounted Display Apparatus:

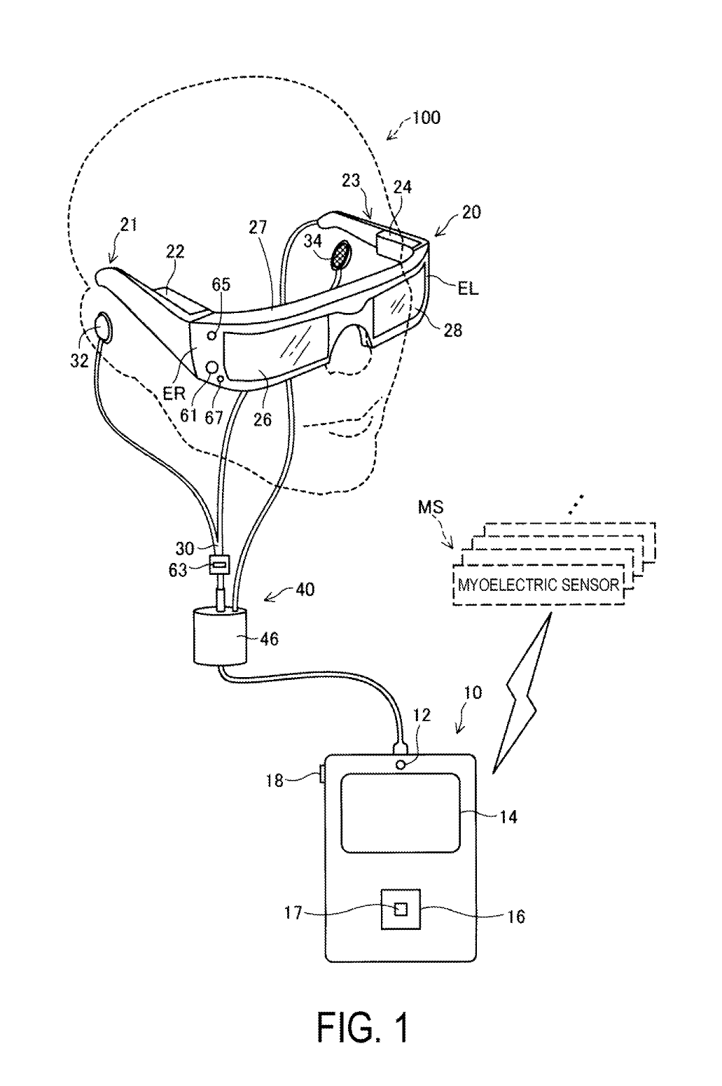

FIG. 1 is an explanatory diagram illustrating a schematic configuration of a transmissive head-mounted display apparatus 100 according to an exemplary embodiment of the present disclosure. The transmissive head-mounted display apparatus 100 is a display apparatus to be mounted on a user's head and is also referred to as a Head-Mounted Display (HMD). The HMD 100 is a see-through (transmissive) head-mounted display apparatus that provides an image appearing in an external scenery viewed through glasses.

In the exemplary embodiment, a user of the HMD 100 attaches the HMD 100 on the head, and also attaches a plurality of myoelectric sensors MS on the arms and the hands to operate the HMD 100. FIG. 1 also illustrates the plurality of myoelectric sensors MS. The myoelectric sensors MS are configured to detect weak electric signals derived from muscle movements of the arms, the thumbs, and the fingers attached with the myoelectric sensors MS. The HMD 100 and the myoelectric sensors MS are coupled with each other in a wireless manner via a wireless communication unit 117, described later. In character input processing, described later, in the exemplary embodiment, results of detection of the myoelectric sensors MS are used, and a hand gesture of the user is recognized. Therefore, a character specified beforehand can be entered in accordance with the hand gesture. The myoelectric sensors MS, hand gestures, and the character input processing will be described later in detail.

The HMD 100 includes an image display unit 20 configured to allow the user to view images and a control device 10 configured to control the image display unit 20.

The image display unit 20 is a head-mounted body to be worn by the user on the head and has an eyeglasses-like shape in the exemplary embodiment. The image display unit 20 includes a support body including a right holding portion 21, a left holding portion 23, and a front frame 27 and further includes, on the support body, a right display unit 22, a left display unit 24, a right light-guiding plate 26, and a left light-guiding plate 28.

The right holding portion 21 and the left holding portion 23 respectively extend rearward from ends of the front frame 27 to hold the image display unit 20 on the user's head in a manner similar to the temples of a pair of eyeglasses. Here, one of both the ends of the front frame 27 located on the right side of the user in a state where the user wears the image display unit 20 is referred to as an end ER, and the other end located on the left side of the user in a state where the user wears the image display unit 20 is referred to as an end EL. The right holding portion 21 is provided to extend from the end ER of the front frame 27 to a position corresponding to the right temple of the user when the user wears the image display unit 20. The left holding portion 23 is provided to extend from the end EL of the front frame 27 to a position corresponding to the left temple of the user when the user wears the image display unit 20.

The right light-guiding plate 26 and the left light-guiding plate 28 are provided in the front frame 27. The right light-guiding plate 26 is positioned in front of the right eye of the user, when the user wears the image display unit 20, to allow the right eye to view an image. The left light-guiding plate 28 is positioned in front of the left eye of the user, when the user wears the image display unit 20, to allow the left eye to view an image.

The front frame 27 has a shape connecting an end of the right light-guiding plate 26 and an end of the left light-guiding plate 28 with each other. The position of connection corresponds to a position between eyebrows of the user when the user wears the image display unit 20. The front frame 27 may include a nose pad portion that is provided at the position of connection between the right light-guiding plate 26 and the left light-guiding plate 28, and that is in contact with the nose of the user when the user wears the image display unit 20. In this case, the nose pad portion, the right holding portion 21, and the left holding portion 23 allow the image display unit 20 to be held on the head of the user. A belt may also be attached to the right holding portion 21 and the left holding portion 23 that fits to the back of the head of the user when the user wears the image display unit 20. In this case, the belt allows the image display unit 20 to be firmly held on the head of the user.

The right display unit 22 is configured to display images on the right light-guiding plate 26. The right display unit 22 is provided on the right holding portion 21 and lies adjacent to the right temple of the user when the user wears the image display unit 20. The left display unit 24 is configured to display images on the left light-guiding plate 28. The left display unit 24 is provided on the left holding portion 23 and lies adjacent to the left temple of the user when the user wears the image display unit 20.

The right light-guiding plate 26 and the left light-guiding plate 28 according to the exemplary embodiment are optical parts (e.g., prisms) formed of a light transmission-type resin or the like, and are configured to guide image light output by the right display unit 22 and the left display unit 24 to the eyes of the user. Surfaces of the right light-guiding plate 26 and the left light-guiding plate 28 may be provided with dimmer plates. The dimmer plates are thin-plate optical elements having a different transmittance for a different wavelength range of light, and function as so-called wavelength filters. The dimmer plates are arranged to cover a surface of the front frame 27 (a surface opposite to a surface facing the eyes of the user), for example. Appropriate selection of optical property of the dimmer plates allows the transmittance of light to a desired wavelength range, such as visible light, infrared light, and ultraviolet light to be adjusted, and allows the amount of outside light entering the right light-guiding plate 26 and the left light-guiding plate 28 and passing through the right light-guiding plate 26 and the left light-guiding plate 28 to be adjusted.

The image display unit 20 guides image light generated by the right display unit 22 and the left display unit 24 to the right light-guiding plate 26 and the left light-guiding plate 28, respectively, to allow the user to view, by the image light, an image (Augmented Reality (AR) image) along with scenery in an outside world viewed through the image display unit 20 (this is also referred to as “display an image”). In a case where the outside light traveling from the front of the user passes through the right light-guiding plate 26 and the left light-guiding plate 28 and enters the eyes of the user, the image light forming an image and the outside light enter the eyes of the user. The visibility of images viewed by the user can be affected by the intensity of the outside light.

The visibility of images may thus be adjusted, for example, by mounting dimmer plates on the front frame 27 and by appropriately selecting or adjusting the optical properties of the dimmer plates. In a typical example, dimmer plates may be selected to have a light transmittance that allows the user with the HMD 100 to view at least an external scenery. The visibility of images may also be improved by suppressing sunlight. The use of the dimmer plates is also expected to be effective in protecting the right light-guiding plate 26 and the left light-guiding plate 28 to prevent, for example, damage and adhesion of dust to the right light-guiding plate 26 and the left light-guiding plate 28. The dimmer plates may be removably attached to the front frame 27 or each of the right light-guiding plate 26 and the left light-guiding plate 28. Alternatively, different types of removable dimmer plates may be provided for replacement, or alternatively the dimmer plates may be omitted.

A camera 61 is arranged on the front frame 27 of the image display unit 20. The camera 61 is provided on a front surface of the front frame 27 and positioned so that the camera 61 does not block the outside light passing through the right light-guiding plate 26 and the left light-guiding plate 28. In the example in FIG. 1, the camera 61 is arranged on the end ER of the front frame 27. The camera 61 may be arranged on the end EL of the front frame 27 or at the connection between the right light-guiding plate 26 and the left light-guiding plate 28.

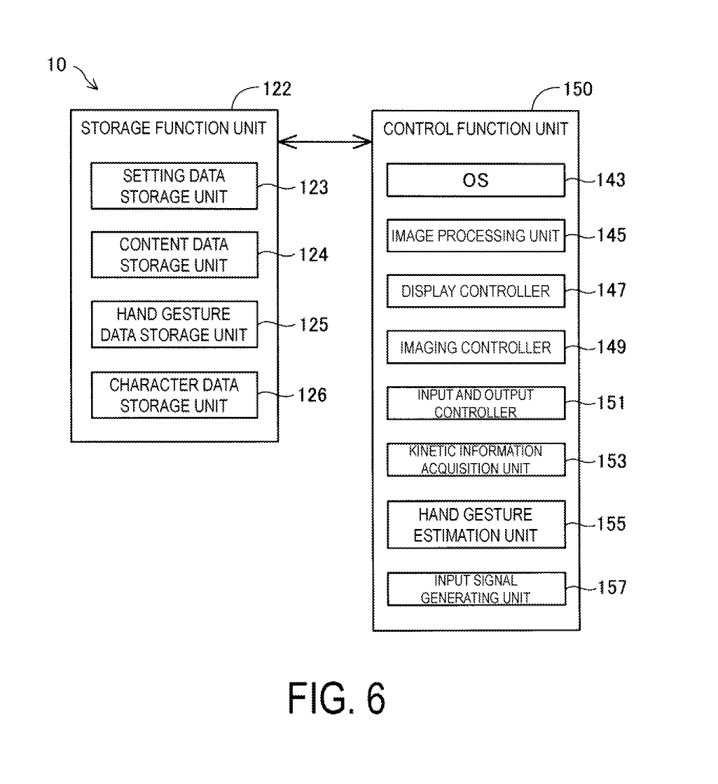

The camera 61 is a digital camera including an imaging lens, and an imaging element such as a charge-coupled device (CCD) and a complementary metal oxide semiconductor (CMOS). The camera 61 according to the exemplary embodiment is a monocular camera. However, a stereo camera may be adopted. The camera 61 is configured to capture an image of at least part of an external scenery (real space) in a front direction of the HMD 100, in other words, in a direction of the field of view of the user when the user wears the image display unit 20. In other words, the camera 61 is configured to capture an image in a range overlapping the field of view of the user or an image in the direction of the field of view of the user, i.e., an image in a direction of a scene viewed by the user. An angle of view of the camera 61 can be appropriately set. In the exemplary embodiment, the angle of view of the camera 61 is set to allow the camera 61 to capture the entire field of view that is visible to the user through the right light-guiding plate 26 and the left light-guiding plate 28. The camera 61 is controlled by a control function unit 150 (FIG. 6) to capture an image and output the data of the captured image to the control function unit 150.

The HMD 100 may include a distance measurement sensor configured to detect the distance to a measured object located along a predetermined measurement direction. The distance measurement sensor may be arranged at the connection between the right light-guiding plate 26 and the left light-guiding plate 28 of the front frame 27, for example. The measurement direction of the distance measurement sensor may be the front direction of the HMD 100 (a direction overlapping an imaging direction of the camera 61). The distance measurement sensor may include, for example, a light emitting part, such as an LED or a laser diode, configured to emit light, and a light receiving part configured to receive light reflected by the object to be measured. In this case, a distance is determined by a triangulation process or a distance measurement process based on a time difference. The distance measurement sensor may include, for example, a transmission part configured to transmit ultrasonic waves and a reception part configured to receive the ultrasonic waves reflected by an object to be measured. In this case, a distance is determined by the distance measurement process based on the time difference. Like the camera 61, the distance measurement sensor measures a distance in accordance with an instruction from the control function unit 150 and outputs the result of detection to the control function unit 150.

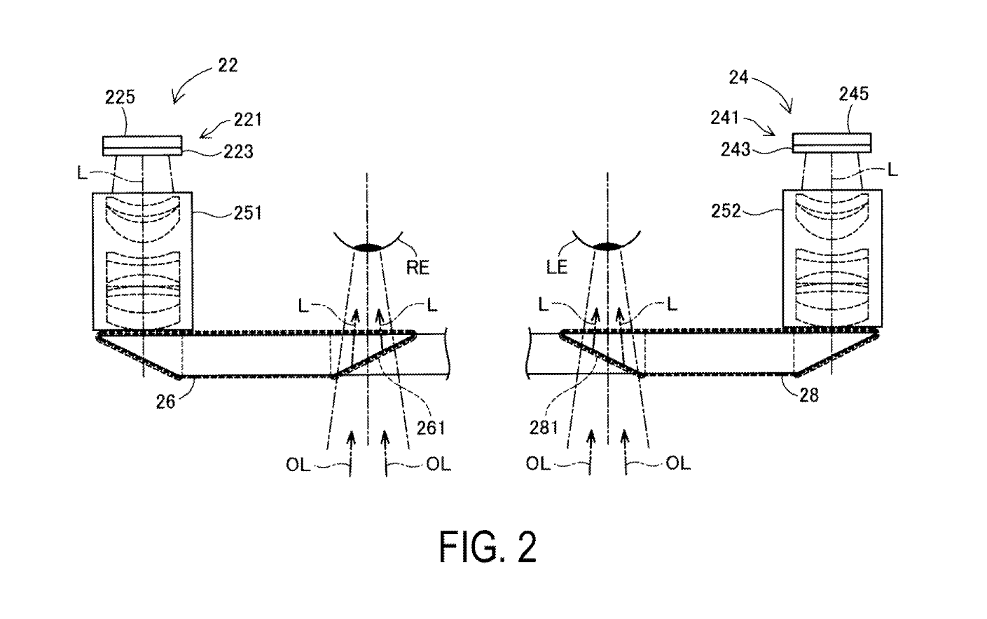

FIG. 2 is a plan view illustrating a main part of a configuration of an optical system included in the image display unit 20. For convenience of description, FIG. 2 illustrates the right eye RE and the left eye LE of the user. As illustrated in FIG. 2, the right display unit 22 and the left display unit 24 are arranged symmetrically on the right- and left-hand sides.

To allow the right eye RE to view an image (AR image), the right display unit 22 includes an organic light emitting diode (OLED) unit 221 and a right optical system 251. The OLED unit 221 is configured to emit imaging light L. The right optical unit 251 includes a lens group and the like and is configured to guide, to the right light-guiding plate 26, imaging light L emitted by the OLED unit 221.

The OLED unit 221 includes an OLED panel 223 and an OLED drive circuit 225 configured to drive the OLED panel 223. The OLED panel 223 is a light emission type display panel including light-emitting elements configured to emit red (R) color light, green (G) color light, and blue (B) color light, respectively, by organic electro-luminescence. The OLED panel 223 includes a plurality of pixels arranged in a matrix, each of the plurality of pixels including one element of R, one element of G, and one element of B.

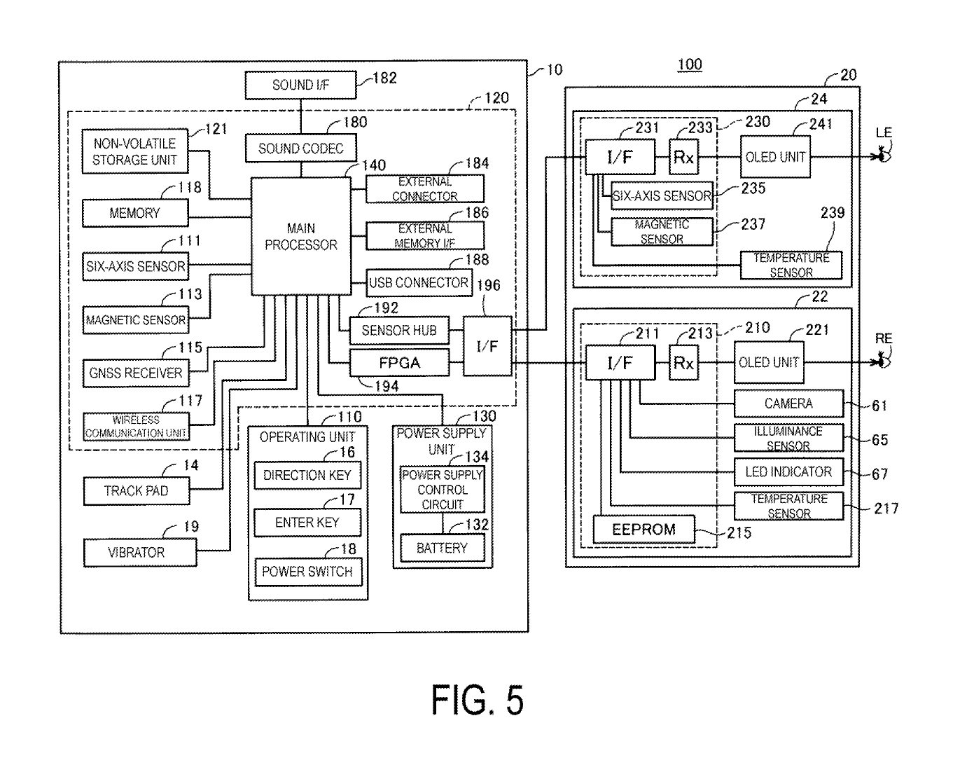

The OLED drive circuit 225 is controlled by the control function unit 150 (FIG. 6), which will be described later, to select and power the light-emitting elements included in the OLED panel 223 to cause the light-emitting elements to emit light. The OLED drive circuit 225 is secured by bonding or the like, for example, onto a rear face of the OLED panel 223, i.e., back of a light-emitting surface. The OLED drive circuit 225 may include, for example, a semiconductor device configured to drive the OLED panel 223, and may be mounted onto a substrate secured to the rear face of the OLED panel 223. A temperature sensor 217 (FIG. 5) described below is mounted on the substrate. The OLED panel 223 may be configured to include light-emitting elements, arranged in a matrix, that emit white color light, and color filters, disposed over the light-emitting elements, that correspond to the R color, the G color, and the B color, respectively. The OLED panel 223 may have a WRGB configuration including light-emitting elements configured to emit white (W) color light, in addition to light-emitting elements configured to emit R color light, G color light, and B color light, respectively.

The right optical system 251 includes a collimate lens configured to collimate the imaging light L emitted from the OLED panel 223. The imaging light L collimated by the collimate lens enters the right light-guiding plate 26. In an optical path configured to guide light inside the right light-guiding plate 26, a plurality of reflective faces configured to reflect the imaging light L is formed. The imaging light L is reflected multiple times inside the right light-guiding plate 26 and then, is guided to the right eye RE side. In the right light-guiding plate 26, a half mirror 261 (reflective face) located in front of the right eye RE is formed. The image light L reflected by the half mirror 261 is emitted from the right light-guiding plate 26 to the right eye RE. The image light L forms an image on the retina of the right eye RE to allow the user to view the image.

To allow the left eye LE to view an image (AR image), the left display unit 24 includes an OLED unit 241 and a left optical system 252. The OLED unit 241 is configured to emit the imaging light L. The left optical system 252 includes a lens group and the like, and is configured to guide, to the left light-guiding plate 28, imaging light L emitted by the OLED unit 241. The OLED unit 241 includes an OLED panel 243 and an OLED drive circuit 245 configured to drive the OLED panel 243. For further details, the OLED unit 241, the OLED panel 243, and the OLED drive circuit 245 are the same as the OLED unit 221, the OLED panel 223, and the OLED drive circuit 225, respectively. A temperature sensor 239 (FIG. 5) is mounted on a substrate secured to a rear face of the OLED panel 243. For further details, the left optical system 252 is the same as the right optical system 251 described above.

According to the configuration described above, the HMD 100 may serve as a see-through display apparatus. That is, the imaging light L reflected by the half mirror 261 and the outside light OL passing through the right light-guiding plate 26 enter the right eye RE of the user. The imaging light L reflected by the half mirror 281 and the outside light OL passing through the left light-guiding plate 28 enter the left eye LE of the user. In this manner, the HMD 100 allows the imaging light L of the internally processed image and the outside light OL to enter the eyes of the user in an overlapped manner. As a result, the user views an external scenery (real world) through the right light-guiding plate 26 and the left light-guiding plate 28 and also views a virtual image (virtual image or AR image) formed by the imaging light L overlapping the external scenery.

The right optical system 251 and the right light-guiding plate 26 are also collectively referred to as a “right light-guiding unit” and the left optical system 252 and the left light-guiding plate 28 are also collectively referred to as a “left light-guiding unit”. Configurations of the right light-guiding unit and the left light-guiding unit are not limited to the example described above, and any desired configuration may be adopted as long as imaging light forms an image in front of the eyes of the user. For example, diffraction gratings or translucent reflective films may be used for the right light-guiding unit and the left light-guiding unit.

In FIG. 1, the control device 10 and the image display unit 20 are connected together via a connection cable 40. The connection cable 40 is removably connected to a connector provided in a lower portion of the control device 10 and connects to various circuits inside the image display unit 20 through a tip of the left holding part 23. The connection cable 40 includes a metal cable or an optical fiber cable through which digital data is transmitted. The connection cable 40 may further include a metal cable through which analog data is transmitted. A connector 46 is provided in the middle of the connection cable 40.

The connector 46 is a jack to which a stereo mini-plug is connected, and is connected to the control device 10, for example, via a line through which analog voice signals are transmitted. In the example of the exemplary embodiment illustrated in FIG. 1, the connector 46 connects to a right earphone 32 and a left earphone 34 constituting a stereo headphone and to a headset 30 including a microphone 63.

As illustrated in FIG. 1, for example, the microphone 63 is arranged such that a sound collector of the microphone 63 faces in a sight direction of the user. The microphone 63 is configured to collect voice and output voice signals to a voice interface 182 (FIG. 5). The microphone 63 may be a monaural microphone or a stereo microphone, or may be a directional microphone or a non-directional microphone.

The control device 10 is used to control the HMD 100. The control device 10 includes an illumination part 12, a track pad 14, a direction key 16, an enter key 17, and a power switch 18. The illumination part 12 is configured to inform the user of an operation state of the HMD 100 (e.g., power ON/OFF) with its light-emitting mode. The illumination part 12 may be, for example, light-emitting diodes (LEDs).

The track pad 14 is configured to detect a touch operation on an operation face of the track pad 14 to output a signal corresponding to what is detected. Any of various track pads, such as an electrostatic-type track pad, a pressure detection-type track pad, and an optical track pad may be adopted as the track pad 14. The direction key 16 is configured to detect a push operation onto any of keys corresponding to up, down, right and left directions to output a signal corresponding to what is detected. The enter key 17 is configured to detect a push operation to output a signal used to determine the operation performed on the control device 10. The power switch 18 is configured to detect a switch sliding operation to switch the state of the power supply for the HMD 100.

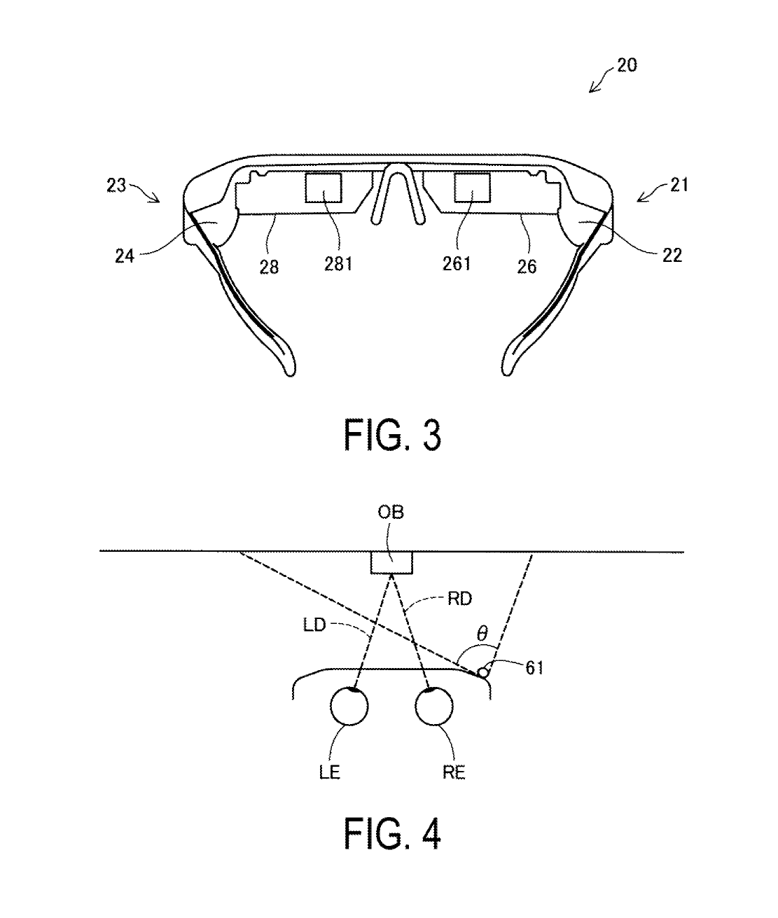

FIG. 3 is a diagram illustrating a configuration of a main part of the image display unit 20 as viewed from the user. In FIG. 3, illustration of the connection cable 40, the right earphone 32, and the left earphone 34 is omitted. In the state illustrated in FIG. 3, back sides of the right light-guiding plate 26 and the left light-guiding plate 28 are visible. The half mirror 261 configured to irradiate the imaging light L to the right eye RE, and the half mirror 281 configured to irradiate imaging light to the left eye LE are also visible as approximately square-shaped regions. The user views an external scenery through the entire areas of the right light-guiding plate 26 and the left light-guiding plate 28 including the half mirrors 261 and 281, and also views rectangular displayed images at the positions of the half mirrors 261 and 281.

FIG. 4 is a diagram illustrating the angle of view of the camera 61. FIG. 4 schematically illustrates the camera 61, along with the right eye RE and left eye LE of the user, in a plan view. The angle of view (imaging range) of the camera 61 is represented by θ. The angle of view θ of the camera 61 extends not only in a horizontal direction as illustrated in the figure, but also in a perpendicular direction as is the case with any common digital camera.

As described above, the camera 61 is arranged at an end ER on the right-hand side of the image display unit 20 to capture an image in the sight direction of the user (i.e., in front of the user). For this purpose, the optical axis of the camera 61 extends in a direction including sight directions of the right eye RE and the left eye LE. The external scenery that is visible when the user wears the HMD 100 is not necessarily an infinitely distant scenery. For example, in a case where the user fixates on an object OB with both eyes, the line-of-sight of the user is directed to the object OB as illustrated by line-of-sights RD and LD in the figure. In this case, the distance from the user to the object OB often ranges from approximately 30 cm to 10 m, both inclusive, and more often ranges from 1 m to 4 m, both inclusive. Thus, standard maximum and minimum distances from the user to the object OB that the user can take during normal use of HMD 100 may be specified. These standards may be predetermined and preset in the HMD 100 or they may be set by the user. The optical axis and the angle of view of the camera 61 are preferably set such that the object OB is included within the angle of view in a case where the distance to the object OB during normal use corresponds to the set standards of the maximum and minimum distances.

In general, the viewing angle of a human is known to be approximately 200 degrees in the horizontal direction and approximately 125 degrees in the vertical direction. Within these angles, an effective visual field advantageous for information acceptance performance is approximately 30 degrees in the horizontal direction and approximately 20 degrees in the vertical direction. In general, a stable field of fixation in which a human can promptly and stably view any point of fixation ranges from approximately 60 degrees to 90 degrees, both inclusive, in the horizontal direction and from approximately 45 degrees to 70 degrees, both inclusive, in the vertical direction. In this case, when the point of fixation lies at the object OB (FIG. 4), the effective visual field is approximately 30 degrees in the horizontal direction and approximately 20 degrees in the vertical direction around the line-of-sights RD and LD. Furthermore, the stable visual field of fixation ranges from approximately 60 degrees to 90 degrees, both inclusive, in the horizontal direction and from approximately 45 degrees to 70 degrees, both inclusive, in the vertical direction. The visual field of the user actually viewing an object through the image display unit 20, the right light-guiding plate 26, and the left light-guiding plate 28 is referred to as an actual field of view (FOV). The actual field of view is narrower than the visual field angle and the stable field of fixation, but is wider than the effective visual field.

The angle of view θ of the camera 61 according to the exemplary embodiment is set to capture a range wider than the visual field of the user. The angle of view θ of the camera 61 is preferably set to capture a range wider than at least the effective visual field of the user and is more preferably set to capture a range wider than the actual field of view. The angle of view θ of the camera 61 is even more preferably set to capture a range wider than the stable field of fixation of the user and is most preferably set to capture a range wider than the visual field angle of the eyes of the user. The camera 61 may thus include a wide angle lens as an imaging lens, and may be configured to capture an image with a wider angle of view. The wide angle lens may include a super-wide angle lens or a semi-wide angle lens. The camera 61 may also include a fixed focal lens, a zoom lens, or a lens group including a plurality of lenses.

FIG. 5 is a functional block diagram illustrating a configuration of the HMD 100. The control device 10 includes a main processor 140 configured to execute a program to control the HMD 100, storages, input and output units, sensors, interfaces, and a power supply unit 130. The main processor 140 connects to the storages, the input/output units, the sensors, the interfaces, and the power supply unit 130. The main processor 140 is mounted on a controller substrate 120 built into the control device 10.

The storages include a memory 118 and a nonvolatile storage 121. The memory 118 constitutes a work area in which computer programs and data to be processed by the main processor 140 are temporarily stored. The non-volatile storage unit 121 is configured by a flash memory or an embedded Multi Media Card (eMMC). The nonvolatile storage unit 121 is configured to store computer programs to be executed by the main processor 140 and various data to be processed by the main processor 140. In the exemplary embodiment, these storages are mounted on the controller substrate 120.

The input and output units include the track pad 14 and an operating unit 110. The operating unit 110 includes the direction key 16, the enter key 17, and the power switch 18, included in the control device 10. The main processor 140 is configured to control the input and output units and acquire signals output from the input and output units.

The sensors include a six-axis sensor 111, a magnetic sensor 113, and a global navigation satellite system (GNSS) receiver 115. The six-axis sensor 111 is a motion sensor (inertia sensor) including a three-axis acceleration sensor and a three-axis gyro (angular velocity) sensor. An inertial measurement unit (IMU) in which these sensors are provided as modules may be adopted as the six-axis sensor 111. The magnetic sensor 113 is a three-axis geomagnetic sensor, for example. The GNSS receiver 115 is configured to determine a present position (longitude and latitude) of the control device 10, based on navigation signals received from an artificial satellite constituting the GNSS. The sensors (six-axis sensor 111, magnetic sensor 113, and GNSS receiver 115) output detected values to the main processor 140 in accordance with a predetermined sampling frequency. The sensors may output detected values at timings instructed by the main processor 140.

The interfaces include a wireless communication unit 117, a voice codec 180, an external connector 184, an external memory interface 186, a universal serial bus (USB) connector 188, a sensor hub 192, a field programmable gate array (FPGA) 194, and an interface 196. The components are configured to function as an interface with external devices.

The wireless communication unit 117 is configured to perform wireless communication between the HMD 100 and an external device. The wireless communication unit 117 is configured to include an antenna (not illustrated), a radio frequency (RF) circuit, a baseband circuit, a communication control circuit, and the like, or is configured as a device into which these components are integrated. The wireless communication unit 117 is configured to perform wireless communication in compliance with standards such as Bluetooth (trade name) and wireless LAN including Wi-Fi (trade name). In the exemplary embodiment, the wireless communication unit 117 performs wireless communications in conformity to Bluetooth (trade name) with the myoelectric sensors MS. Instead of Bluetooth (trade name), near-field wireless communications in conformity to Near Field Communication (NFC), Felica (trade name), or radio frequency identification (RFID), or a wireless local area network (LAN) conforming to IEEE802.11a/b/g/n/ac, for example, may be used.

The voice codec 180 is connected to the voice interface 182 and is configured to encode and decode voice signals input and output via the voice interface 182. The voice interface 182 is an interface configured to input and output the voice signals. The voice codec 180 may include an A/D converter configured to convert an analog voice signal into digital voice data and a digital/analog (D/A) converter configured to convert digital voice data into an analog voice signal. The HMD 100 according to the exemplary embodiment outputs voice from the right earphone 32 and the left earphone 34 and collects voice from the microphone 63. The voice codec 180 is configured to convert digital voice data output by the main processor 140 into an analog voice signal, and output the analog voice signal via the voice interface 182. The voice codec 180 is also configured to convert an analog voice signal input to the voice interface 182 into digital voice data, and output the digital voice data to the main processor 140.

The external connector 184 is a connector configured to connect the main processor 140 to an external device (e.g., personal computer, smartphone, or gaming device) configured to communicate with the main processor 140. The external device connected to the external connector 184 may serve as a source of content, may debug a computer program to be executed by the main processor 140, and may collect an operation log of the HMD 100. The external connector 184 may take various forms. The external connector 184 may be a wired-connection interface such as a USB interface, a micro USB interface, and memory card interface, or a wireless-connection interface such as a wireless LAN interface and a Bluetooth interface.

The external memory interface 186 is an interface configured to connect a portable memory device. The external memory interfaces 186 include, for example, a memory card slot configured to accept a card recording medium for reading and writing data, and an interface circuit. For example, the size and shape of the card recording medium, as well as standards to be used for the card recording medium, may be appropriately selected. The USB connector 188 is an interface configured to connect a memory device, a smartphone, a personal computer, or the like in compliance with the USB standard. The USB connector 188 includes, for example, a connector and an interface circuit in compliance with the USB standard. For example, the size and shape of the USB connector 188, as well as the version of USB standard to be used for the USB connector 188, may be appropriately selected.

The HMD 100 further includes a vibrator 19. The vibrator 19 includes a motor (not illustrated), an eccentric rotor, and the like, and is configured to generate vibration under the control of the main processor 140. The HMD 100 causes the vibrator 19 to generate vibration in a predetermined vibration pattern, for example, in a case where an operation on the operation unit 110 is detected, or in a case where a power supply of the HMD 100 is turned on or off. The vibrator 19 may be provided, instead of being provided in the control device 10, in the image display unit 20, for example, in the right holding part 21 (right temple side) of the image display unit 20.

The sensor hub 192 and the FPGA 194 are connected to the image display unit 20 via the interface (I/F) 196. The sensor hub 192 is configured to acquire detected values of the sensors included in the image display unit 20 and output the detected values to the main processor 140. The FPGA 194 is configured to process data to be transmitted and received between the main processor 140 and components of the image display unit 20, and perform transmissions via the interface 196. The interface 196 is connected to the right display unit 22 and the left display unit 24 of the image display unit 20. In the example of the exemplary embodiment, the connection cable 40 is connected to the left holding part 23. Wiring, in the image display unit 20, connected to the connection cable 40 causes the right display unit 22 and the left display unit 24 to be connected to the interface 196 of the control device 10.

The power supply unit 130 includes a battery 132 and a power supply control circuit 134. The power supply unit 130 is configured to supply power used to operate the control device 10. The battery 132 is a rechargeable battery. The power supply control circuit 134 is configured to detect a remaining capacity of the battery 132 and control charging of an operating system (OS) 143 (FIG. 6). The power supply control circuit 134 is connected to the main processor 140, and is configured to output the detected value of the remaining capacity of the battery 132 and the detected value of a voltage of the battery 132 to the main processor 140. Power may be supplied from the control device 10 to the image display unit 20, based on the power supplied by the power supply unit 130. The main processor 140 may be configured to control the state of power supply from the power supply unit 130 to components of the control device 10 and the image display unit 20.

The right display unit 22 includes a display unit substrate 210, an OLED unit 221, a camera 61, an illuminance sensor 65, an LED indicator 67, and a temperature sensor 217. The display unit substrate 210 is equipped with an interface (I/F) 211 connected to the interface 196, a receiving unit (Rx) 213, and an electrically erasable programmable read-only memory (EEPROM) 215. The receiving unit 213 is configured to receive data from the control device 10 via the interface 211. In a case of receiving image data of an image to be displayed on the OLED unit 221, the receiving unit 213 outputs the received image data to the OLED drive circuit 225 (FIG. 2).

The EEPROM 215 is configured to store various data in such a manner that the main processor 140 can read the data. The EEPROM 215 is configured to store, for example, data about light emission properties and display properties of the OLED units 221 and 241 of the image display unit 20, and data about sensor properties of the right display unit 22 or the left display unit 24. Specifically, for example, the EEPROM 215 is configured to store parameters regarding Gamma correction performed by the OLED units 221 and 241, and data used to compensate for the detected values of the temperature sensors 217 and 239 described below. These kinds of data are generated by inspection at the time of shipping of the HMD 100 from a factory, and are written into the EEPROM 215. After shipment, the data is loaded from the EEPROM 215 into the main processor 140, and is used for various processes.

The camera 61 is configured to capture an image in accordance with a signal entered via the interface 211, and output imaging data or a signal indicating the result of imaging to the control device 10. As illustrated in FIG. 1, the illuminance sensor 65 is arranged on the end ER of the front frame 27 and is configured to receive outside light from the front of the user wearing the image display unit 20. The illuminance sensor 65 is configured to output a detected value corresponding to the amount of received light (intensity of received light). As illustrated in FIG. 1, the LED indicator 67 is disposed near the camera 61 at the end ER of the front frame 27. The LED indicator 67 is configured to be turned on during image capturing by the camera 61 to notify that the image capturing is in progress.

The temperature sensor 217 is configured to detect a temperature to output a voltage value or a resistance value corresponding to the detected temperature. The temperature sensor 217 is mounted on the rear face of the OLED panel 223 (FIG. 2). The temperature sensor 217 may be mounted, for example, on the same substrate as the substrate on which the OLED drive circuit 225 is mounted. This configuration allows the temperature sensor 217 to mainly detect the temperature of the OLED panel 223. The temperature sensor 217 may be built into the OLED panel 223 or the OLED drive circuit 225 (FIG. 2). For example, in a case where the OLED panel 223, together with the OLED drive circuit 225, is mounted as an Si-OLED on an integrated semiconductor chip to form an integrated circuit, the temperature sensor 217 may be mounted on the semiconductor chip.

The left display unit 24 includes a display unit substrate 230, an OLED unit 241, and a temperature sensor 239. The display unit substrate 230 is equipped with an interface (I/F) 231 connected to the interface 196, a receiving unit (Rx) 233, a 6-axis sensor 235, and a magnetic sensor 237. The receiving unit 233 is configured to receive data input from the control device 10 via the interface 231. In a case where the receiving unit 233 receives image data of an image to be displayed on the OLED unit 241, the receiving unit 233 outputs the received image data to the OLED drive circuit 245 (FIG. 2).

The six-axis sensor 235 is a motion sensor (inertial sensor) including a three-axis acceleration sensor and a three-axis gyro (angular velocity) sensor. The six-axis sensor 235 may be an IMU in which the above-described sensors are provided as modules. The magnetic sensor 237 is a three-axis geomagnetic sensor, for example. The six-axis sensor 235 and the magnetic sensor 237 are provided in the image display unit 20, and thus detect a motion of the head of the user when the image display unit 20 is mounted on the user's head. The orientation of the image display unit 20, i.e., the field of view of the user, is determined based on the detected motion of the head.

The temperature sensor 239 is configured to detect the temperature to output a voltage value or a resistance value corresponding to the detected temperature. The temperature sensor 239 is mounted on the rear face of the OLED panel 243 (FIG. 2). The temperature sensor 239 may be mounted, for example, on the same substrate as the substrate on which the OLED drive circuit 245 is mounted. This configuration allows the temperature sensor 239 to mainly detect the temperature of the OLED panel 243. The temperature sensor 239 may be built into the OLED panel 243 or the OLED drive circuit 245 (FIG. 2). Details about the temperature sensor 239 are identical to the details about the temperature sensor 217.

The sensor hub 192 of the control device 10 connects to the camera 61, the illuminance sensor 65, and the temperature sensor 217 of the right display unit 22, and to the six-axis sensor 235, the magnetic sensor 237, and the temperature sensor 239 of the left display unit 24. The sensor hub 192 is configured to set and initialize a sampling period of each sensor under the control of the main processor 140. Based on the sampling periods of the sensors, the sensor hub 192 supplies power to the sensors, transmits control data, and acquires detected values, for example. The sensor hub 192 is configured to output, at a preset timing, detected values of the sensors included in the right display unit 22 and the left display unit 24, to the main processor 140. The sensor hub 192 may be configured to include a cache function to temporarily retain the detected values of the sensors. The sensor hub 192 may be configured to include a function to convert a signal format or a data format of detected values of the sensors (e.g., function for conversion into a standard format). The sensor hub 192 is configured to start and stop supplying power to the LED indicator 67 under the control of the main processor 140 to turn on or off the LED indicator 67.

FIG. 6 is a functional block diagram illustrating a configuration of the control device 10. In terms of functions, the control device 10 includes a storage function unit 122 and a control function unit 150. The storage function unit 122 is a logical storage configured based on the nonvolatile storage 121 (FIG. 5). Instead of a configuration in which only the storage function unit 122 is used, the storage function unit 122 may be configured to use the EEPROM 215 or the memory 118 in combination with the nonvolatile storage 121. The control function unit 150 is configured upon the main processor 140 that executes a computer program, i.e., upon hardware and software that operate together.

The storage function unit 122 is configured to store various data to be processed by the control function unit 150. Specifically, the storage function unit 122 according to the exemplary embodiment includes a setting data storage unit 123, a content data storage unit 124, a hand gesture data storage unit 125, and a character data storage unit 126. The setting data storage unit 123 is configured to store various setting values regarding operations of the HMD 100. For example, the setting data storage unit 123 is configured to store parameters, determinants, arithmetic expressions, look up tables (LUTs), and the like that are used by the control function unit 150 for control of the HMD 100.

The content data storage unit 124 is configured to store data (image data, video data, voice data, and the like) of contents including images and videos to be displayed by the image display unit 20 under the control of the control function unit 150. The content data storage unit 124 may be configured to store data of bidirectional content. The bidirectional content means a type of content that is displayed by the image display unit 20 in accordance with an operation of the user. The operating unit 110 acquires the operation of the user, the control function unit 150 performs a process corresponding to the acquired operation, and the image display unit 20 displays a content corresponding to the process. In this case, the data of content may include data such as image data of a menu screen used to acquire an operation of the user, and data for specifying a process corresponding to an item included in the menu screen.

The hand gesture data storage unit 125 is stored with information (hereinafter referred to as “kinetic information”) about kinds of hand gestures and muscle movements of arms and hands associated with each other beforehand. “Hand gestures” denote movements of arms, whole hands, and parts of the arms and the hands, such as tips of thumbs and fingers and palms, and, in the exemplary embodiment, movements of hands, thumbs, and fingers used for sign language. Furthermore, the “kinds of hand gestures” denote how the arms and the hands are moved, such as a gesture of waving or closing a hand, and, in the exemplary embodiment, kinds of sign language operations.

In the exemplary embodiment, the “kinetic information” denotes information indicative of a potential at a skin surface detected by one of the myoelectric sensors MS when a muscle movement occurs on an arm or a hand. The “muscle movements” denote, for example, muscle movements when a hand is closed and a hand muscle is strained, and muscular movements when muscles contract and relax, as well as refer to a wide concept including minute muscle activities when cells and tissues, such as nerves, are excited. The kinetic information includes information indicative of myoelectric waveforms indicative of temporal changes in myoelectric potential within a predetermined time and a maximum myoelectric potential, for example. The kinetic information is measured beforehand through experiments per kind of hand gesture, associated with the kind of hand gesture, and stored. The kinds of hand gestures and the kinetic information are used in the character input processing, described later, to estimate a hand gesture performed by a user of the HMD 100.

The character data storage unit 126 is stored with the kinds of hand gestures and characters corresponding to the hand gestures associated with each other beforehand. In the exemplary embodiment, the “characters corresponding to hand gestures” denote characters associated with movements of hands, thumbs, and fingers used for sign language, and are specified beforehand for the kinds of hand gestures.

The control function unit 150 is configured to use the data stored in the storage function unit 122 to execute various processes, thereby performing functions of the operating system (OS) 143, an image processing unit 145, a display controller 147, an imaging controller 149, an input and output controller 151, a kinetic information acquisition unit 153, a hand gesture estimation unit 155, and an input signal generating unit 157. In the exemplary embodiment, the function units other than the OS 143 are configured as computer programs to be executed on the OS 143.

The image processing unit 145 is configured to generate, based on image data or video data to be displayed on the image display unit 20, signals to be transmitted to the right display unit 22 and the left display unit 24. The signals generated by the image processing unit 145 may be a vertical synchronization signal, a horizontal synchronization signal, a clock signal, an analog image signal, and the like. The image processing unit 145 may be implemented by the main processor 140 that executes a corresponding computer program, or may be configured by using hardware different from the main processor 140 (e.g., digital signal processor (DSP)).

The image processing unit 145 may be configured to execute a resolution conversion process, an image adjustment process, a 2D/3D conversion process, and the like as needed. The resolution conversion process is a process for converting the resolution of image data into a resolution appropriate for the right display unit 22 and the left display unit 24. The image adjustment process is a process for adjusting the brightness and saturation of image data. The 2D/3D conversion process is a process for generating two-dimensional image data from three-dimensional image data, or generating three-dimensional image data from two-dimensional image data. In a case where any of the processes is executed, the image processing unit 145 is configured to generate a signal for displaying an image based on the processed image data and transmits the signal to the image display unit 20 via the connection cable 40.

The display controller 147 is configured to generate control signals for controlling the right display unit 22 and the left display unit 24, and use the control signals to control the generation and emission of the image light L by each of the right display unit 22 and the left display unit 24. Specifically, the display controller 147 is configured to control the OLED drive circuits 225 and 245 to cause the OLED panels 223 and 243 to display images. The display controller 147 is configured to control, for example, a timing when the OLED drive circuits 225 and 245 draw images on the OLED panels 223 and 243, and brightness of the OLED panels 223 and 243, based on the signal output by the image processing unit 145. The display controller 147 is configured to cause the image display unit 20 to display, in the character input processing, described later, display images representing hand gestures (hereinafter referred to as “hand gesture display images”) and display images representing the characters corresponding to the hand gestures (hereinafter referred to as “character display images”).

The imaging controller 149 is configured to control the camera 61 to capture an image and generate captured imaging data, and to cause the storage function unit 122 to temporarily store the captured imaging data. In a case where the camera 61 is configured as a camera unit including a circuit for generating imaging data, the imaging controller 149 is configured to acquire the imaging data from the camera 61 and causes the storage function unit 122 to temporarily store the imaging data.

The input and output controller 151 is configured to appropriately control the track pad 14 (FIG. 1), the direction key 16, and the enter key 17 to receive input commands. The received input commands are output to the OS 143 or to a computer program that operates on the OS 143 together with the OS 143. The input and output controller 151 is configured to receive, in the character input processing, described later, an input signal generated by the input signal generating unit 157.

The kinetic information acquisition unit 153 is configured to acquire kinetic information described above from the myoelectric sensors MS via the wireless communication unit 117.

The hand gesture estimation unit 155 is configured to use the kinetic information to estimate a hand gesture. Specifically, the hand gesture estimation unit 155 cross-checks a myoelectric waveform acquired from kinetic information with the kinetic information stored beforehand in the hand gesture data storage unit 125 to estimate a kind of hand gesture. The hand gesture estimation unit 155 may use a pattern recognition method to identify from a myoelectric waveform a movement of a hand, a thumb, or a finger, cross-check the movement being identified with the kinds of hand gestures stored in the hand gesture data storage unit 125, and estimate a hand gesture. A method for estimating a hand gesture will be described later in detail.

The input signal generating unit 157 is configured to generate an input signal about a character corresponding to the hand gesture being estimated and output the input signal being generated to the input and output controller 151. In the exemplary embodiment, when a hand gesture being estimated corresponds to a hand gesture indicative of “determination of input signal corresponding to hand gesture estimated already” (hereinafter referred to as “determination of input signal”), the input signal generating unit 157 does not generate an input signal about a character corresponding to the hand gesture, but generates and outputs an input signal about a character corresponding to a hand gesture estimated already before the hand gesture indicative of “determination of input signal” is performed.

In the exemplary embodiment, the camera 61 corresponds to a subordinate concept of an imaging unit in other exemplary embodiments. The myoelectric sensors MS correspond to subordinate concepts of muscle activity detection devices in other exemplary embodiments.

A2. Augmented Reality Display:

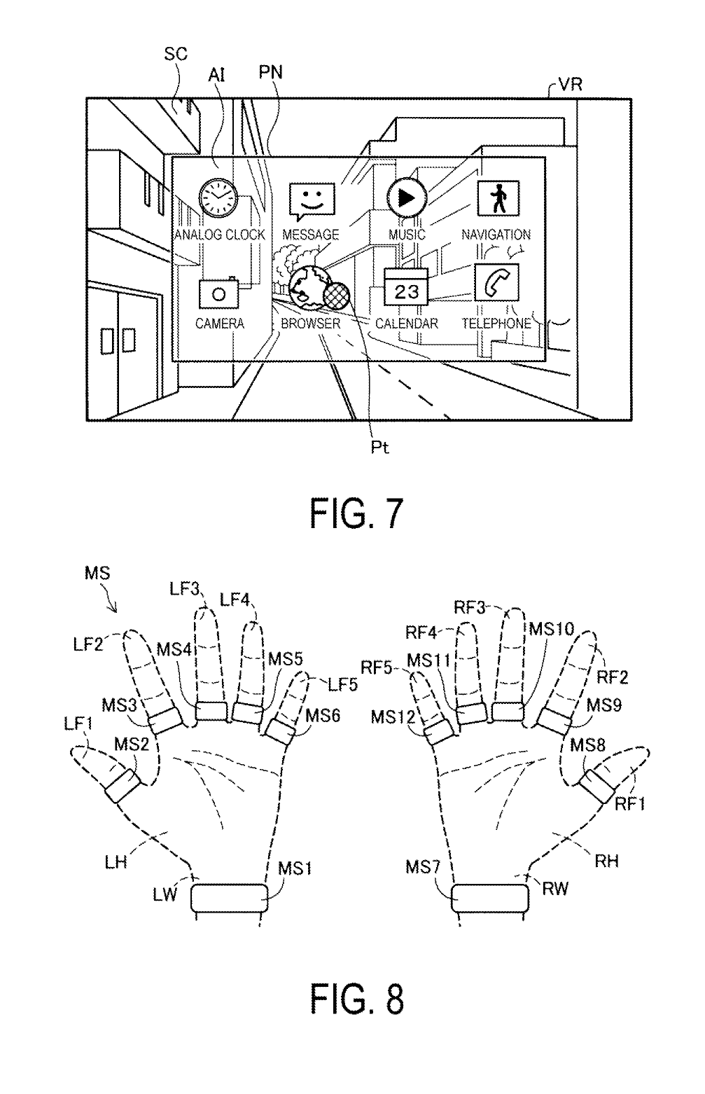

FIG. 7 is an explanatory diagram illustrating an example of augmented reality display provided by the HMD 100. FIG. 7 illustrates the field of view VR of the user. As described above, the image light L guided to the eyes of the user of the HMD 100 is formed into an image on the retinas of the user, allowing the user to view, in the display region PN, an object image AI of a display object as augmented reality (AR). In the example illustrated in FIG. 7, the object image AI is a menu screen of the OS of the HMD 100. The menu screen includes icon images for activating application programs such as “Analog clock”, “Message”, “Music”, “Navigation”, “Camera”, “Browser”, “Calendar”, and “Telephone”. Furthermore, an external light passes through the right light-guiding plate 26 and the left light-guiding plate 28, allowing the user to view an external scenery SC. Thus, the user of the HMD 100 according to the exemplary embodiment can view, in a portion displaying the object image AI in the field of view VR, the object image AI in such a manner that the object image AI overlaps the external scenery SC. Furthermore, the user of the HMD 100 according to the exemplary embodiment can view, in a portion not displaying the object image AI in the field of view VR, only the external scenery SC.

As illustrated in FIG. 7, a pointer image Pt is displayed on the object image AI. The pointer image Pt is used by the user to select each menu displayed on the object image AI. In the example illustrated in FIG. 7, the user moves the pointer image Pt onto the “Browser” icon image on the object image AI to select the “Browser” menu. In this state, the user can tap on the track pad 14 to run the “Browser” menu.

A3. Character Input Processing:

FIG. 8 is an explanatory diagram schematically illustrating the myoelectric sensors MS attached to the hands and the arms of the user. In FIG. 8, the hands and the arms of the user are rendered with dashed lines, whereas the myoelectric sensors MS are rendered with solid lines. As illustrated in FIG. 8, a first myoelectric sensor MS1 is attached to a left arm LW of a left hand LH. A second myoelectric sensor MS2 is attached to a thumb LF1 of the left hand LH, a third myoelectric sensor MS3 is attached to an index finger LF2, a fourth myoelectric sensor MS4 is attached to a third finger LF3, a fifth myoelectric sensor MS5 is attached to a fourth finger LF4, and a sixth myoelectric sensor MS6 is attached to a fifth finger LF5. Similarly, a seventh myoelectric sensor MS7 is attached to a right arm RW of a right hand RH. An eighth myoelectric sensor MS8 is attached to a thumb RF1 of the right hand RH, a ninth myoelectric sensor MS9 is attached to an index finger RF2, a tenth myoelectric sensor MS10 is attached to a third finger RF3, an eleventh myoelectric sensor MS11 is attached to a fourth finger RF4, and a twelfth myoelectric sensor MS12 is attached to a fifth finger RF5.

The myoelectric sensors MS1 to MS12 detect weak electric signals derived from muscle movements of the arms, the thumbs, and the fingers attached with the myoelectric sensors MS1 to MS12, as well as detect a potential at a skin surface when a muscle movement occurs. The myoelectric sensors MS1 and MS7 are arm ring type myoelectric sensors and arranged with surface electrodes on surfaces facing the arms LW and RW. The myoelectric sensors MS2 to MS6 and MS8 to MS12 are ring type myoelectric sensors and arranged with surface electrodes on surfaces facing the thumbs and fingers LF1 to LF5 and RF1 to RF5. The myoelectric sensors MS1 to MS12 include wireless communication units (not illustrated), use the wireless communication units to perform wireless communications in conformity to Bluetooth (trade name) with the wireless communication unit 117, and send myoelectric potentials being detected to the kinetic information acquisition unit 153. The kinetic information acquisition unit 153 is configured to acquire the myoelectric potentials being detected, through wireless communications in conformity to Bluetooth (trade name).

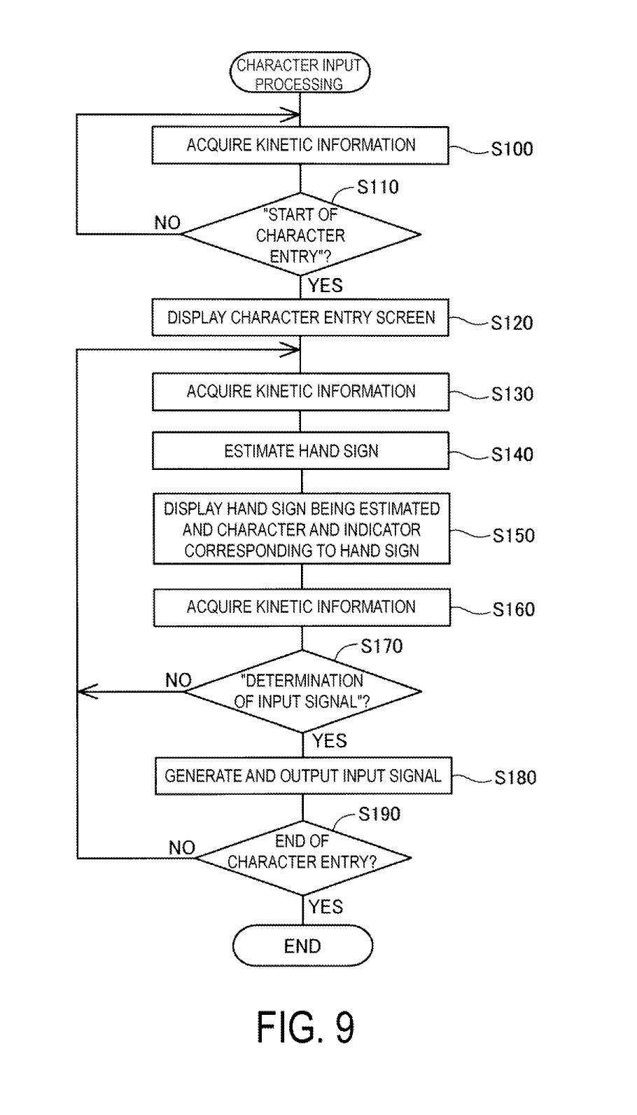

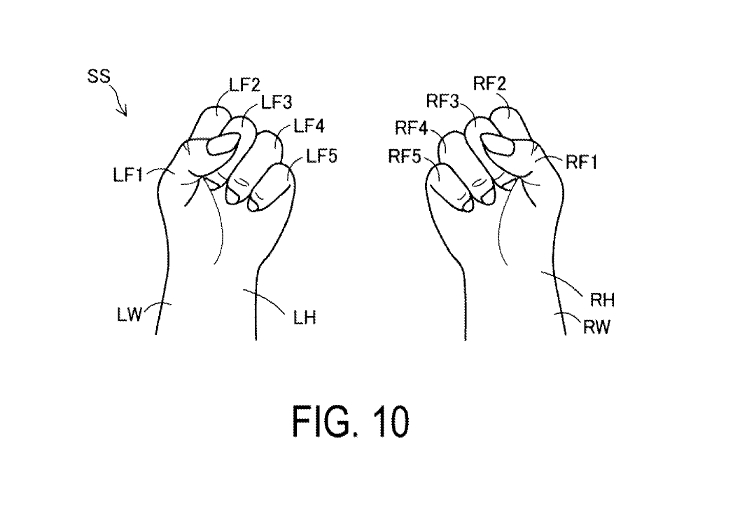

FIG. 9 is a flowchart illustrating how the character input processing proceeds. As the “Browser” application illustrated in FIG. 7 starts, the character input processing starts. As illustrated in FIG. 9, the kinetic information acquisition unit 153 acquires kinetic information from the myoelectric sensors MS1 to MS12 via the wireless communication unit 117 (step S100).

The hand gesture estimation unit 155 estimates a hand gesture from the kinetic information being acquired and determines whether the hand gesture being estimated corresponds to a hand gesture indicative of “start of character entry” (step S110).

FIG. 10 is an explanatory diagram schematically illustrating a hand gesture SS indicative of “start of character entry”. In FIG. 10, illustration of the myoelectric sensors MS1 to MS12 illustrated in FIG. 8 is omitted. As illustrated in FIG. 10, in the exemplary embodiment, the hand gesture SS indicative of “start of character entry” denotes that the left hand LH and the right hand RH of the user of the HMD 100 are closed for a predetermined time (the hand shapes are referred to as “guu (fist shape)” in Japan). Whether the left hand LH and the right hand RH are closed can be identified by determining whether myoelectric potentials equal to or above a predetermined threshold value are detected by the myoelectric sensors MS1 to MS12 for a predetermined time. The predetermined time is one second. The predetermined time may be another desired time than one second.

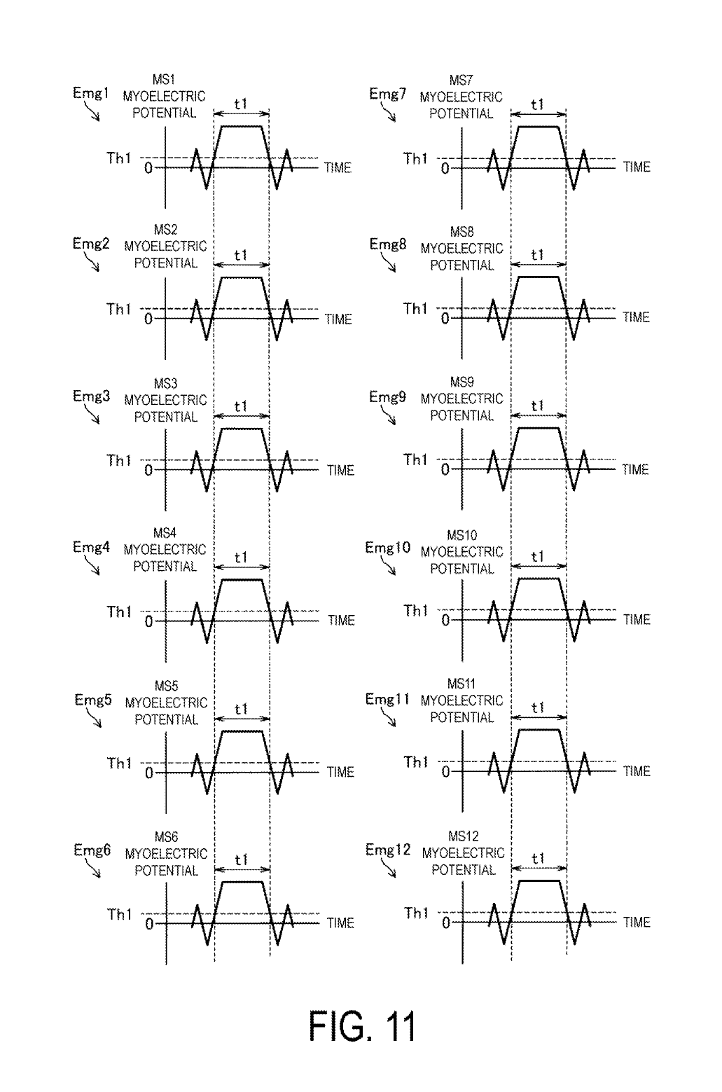

FIG. 11 is an explanatory diagram schematically illustrating temporal changes in myoelectric potential when the hand gesture SS indicative of “start of character entry” is performed. FIG. 11 illustrates myoelectric waveforms Emg1 to Emg12 acquired from results of detection by the myoelectric sensors MS1 to MS12 for the predetermined time. FIG. 11 illustrates, on the left, the myoelectric waveform Emg1 to Emg6 acquired from the results of detection by the myoelectric sensors MS1 to MS6 attached to the left hand LH. FIG. 11 illustrates, on the right, the myoelectric waveform Emg7 to Emg12 acquired from the results of detection by the myoelectric sensor MS7 to MS12 attached to the right hand RH.

Specifically, the top on the left in FIG. 11 illustrates the myoelectric waveform Emg1 of the first myoelectric sensor MS1 attached to the left arm LW of the left hand LH. The second row from the top illustrates the myoelectric waveform Emg2 of the second myoelectric sensor MS2 attached to the thumb LF1 of the left hand LH. The third row from the top illustrates the myoelectric waveform Emg3 of the third myoelectric sensor MS3 attached to the index finger LF2 of the left hand LH. The fourth row from the top illustrates the myoelectric waveform Emg4 of the fourth myoelectric sensor MS4 attached to the third finger LF3 of the left hand LH. The fifth row from the top illustrates the myoelectric waveform Emg5 of the fifth myoelectric sensor MS5 attached to the fourth finger LF4 of the left hand LH. The sixth row from the top illustrates the myoelectric waveform Emg6 of the sixth myoelectric sensor MS6 attached to the fifth finger LF5 of the left hand LH.

The top on the right in FIG. 11 illustrates the myoelectric waveform Emg7 of the seventh myoelectric sensor MS7 attached to the right arm RW of the right hand RH. The second row from the top illustrates the myoelectric waveform Emg8 of the eighth myoelectric sensor MS8 attached to the thumb RF1 of the right hand RH. The third row from the top illustrates the myoelectric waveform Emg9 of the ninth myoelectric sensor MS9 attached to the index finger RF2 of the right hand RH. The fourth row from the top illustrates the myoelectric waveform Emg10 of the tenth myoelectric sensor MS10 attached to the third finger RF3 of the right hand RH. The fifth row from the top illustrates the myoelectric waveform Emg11 of the eleventh myoelectric sensor MS11 attached to the fourth finger RF4 of the right hand RH. The sixth row from the top illustrates the myoelectric waveform Emg12 of the twelfth myoelectric sensor MS12 attached to the fifth finger RF5 of the right hand RH.

In the myoelectric waveforms Emg1 to Emg12, vertical axes indicate the myoelectric potentials detected by the myoelectric sensors MS1 to MS12, whereas horizontal axes indicate time. The myoelectric waveforms Emg1 to Emg12 have similar shapes. Specifically, during a time t1, a myoelectric potential is equal to or above a first threshold value Th1. This is due to the fact that, since the left hand LH and the right hand RH are both closed in the hand gesture SS indicative of “start of character entry”, the arms LW and RW and the thumbs and fingers LF1 to LF5 and RF1 to RF5 are squeezed almost evenly. In the example illustrated in FIG. 11, when the time t1 described above is equal to or above one second, the hand gesture SS indicative of “start of character entry” is estimated. The first threshold value Th1 is 30 microvolts [pV], for example. The first threshold value Th1 may be set to another desired value than 30 microvolts [pV].

In step S110 described above, the hand gesture estimation unit 155 performs pattern matching between shapes of the myoelectric waveforms Emg1 to Emg12 acquired from the kinetic information acquisition unit 153 and a shape of a myoelectric waveform (hereinafter referred to as “reference waveform”) stored in the hand gesture data storage unit 125. The hand gesture estimation unit 155 determines whether the myoelectric sensors MS1 to MS12 detect myoelectric potentials each equal to or above the first threshold value Th1 for the predetermined time (one second) or longer. Why the hand gesture estimation unit 155 checks a value of a myoelectric potential in addition to a myoelectric waveform is to suppress an error or noise from being detected, as well as suppress a wrong hand gesture from being estimated, since an ordinary surface myoelectric potential ranges from several microvolts [pV] to several ten millivolts [mV]. The hand gesture estimation unit 155 may sequentially determine myoelectric potentials of the myoelectric sensors MS1 to MS12, or may perform pattern matching to determine whether shapes of the myoelectric waveforms Emg1 to Emg12 and the shape of the reference waveform each match each other, and then determine whether the myoelectric potentials are each equal to or above the first threshold value Th1.

When neither the shapes of the myoelectric waveforms Emg1 to Emg12 and the shape of the reference waveform each match each other nor a myoelectric potential greater than the first threshold value Th1 is detected for the predetermined time (one second) or longer, the hand gesture estimation unit 155 determines that the hand gesture being estimated does not correspond to the hand gesture SS indicative of “start of character entry” (step S110: NO). Step S100 described above is then executed. On the other hand, when the shapes of the myoelectric waveforms Emg1 to Emg12 and the shape of the reference waveform each match each other, and a myoelectric potential greater than the first threshold value Th1 is detected for the predetermined time (one second) or longer, the hand gesture estimation unit 155 determines that the hand gesture being estimated corresponds to the hand gesture SS indicative of “start of character entry” (step S110: YES). The display controller 147 then causes the character entry screen to appear (step S120).

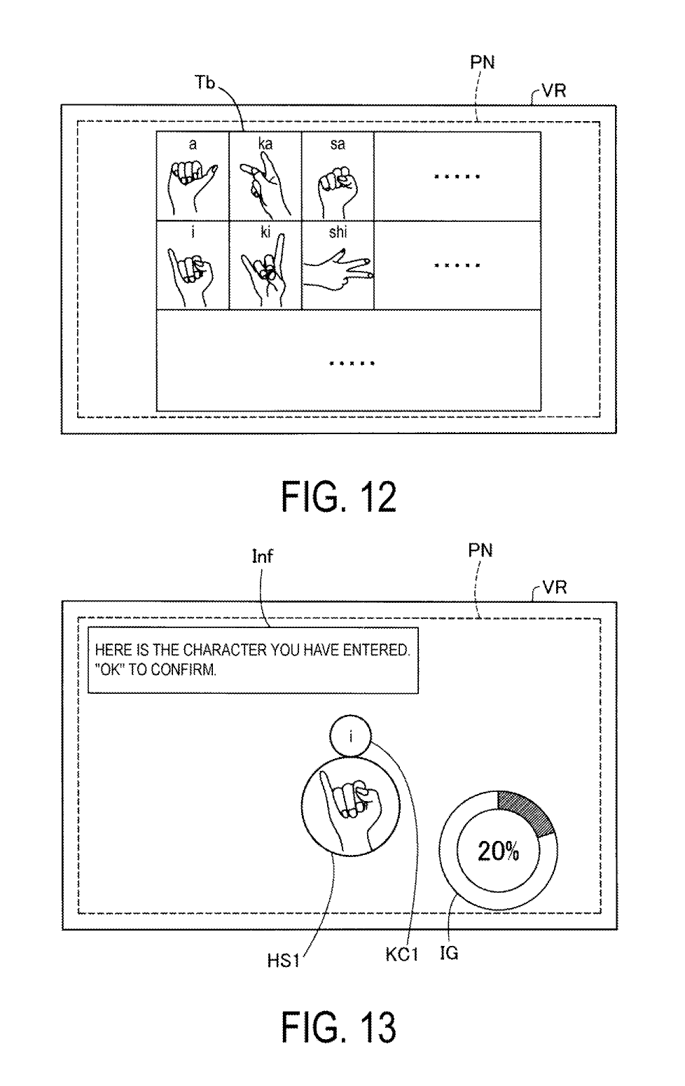

FIG. 12 is an explanatory diagram schematically illustrating the field of view VR of the user after execution of step S120. The external scenery SC is omitted in FIG. 12. As illustrated in FIG. 12, the display region PN displays a model image (hereinafter referred to as “finger spelling display image”) Tb representing a finger spelling display table. The finger spelling display image Tb displays finger spellings and text characters corresponding to the finger spellings associated with each other beforehand. The example illustrated in FIG. 12 displays, in a leftmost column, in an order from upper to lower, a text character “a” and a finger spelling corresponding to the character “a” and a text character “i” and a finger spelling corresponding to the character “i”. Similarly, in a column to right of the leftmost column and still another column to further right of the leftmost column, text characters and finger spellings corresponding to the characters are displayed. By displaying the finger spelling display image Tb as described above, the user can precisely execute a hand gesture.

As illustrated in FIG. 9, the kinetic information acquisition unit 153 acquires kinetic information (step S130). Step S130 is similar to step S100 described above, and its detailed description is omitted. The hand gesture estimation unit 155 estimates a hand gesture (step S140). Specifically, similar to step S110 described above, the hand gesture estimation unit 155 performs pattern matching on a myoelectric waveform to estimate a kind of a hand gesture corresponding to the myoelectric waveform. At this time, the hand gesture estimation unit 155 may extract feature values from the myoelectric potentials being acquired, determine a posture of the hand, and narrow down a hand gesture. Known techniques can be used to achieve a method for extracting a feature value from a myoelectric potential and a method for determining a posture of a hand from the feature value. The hand gesture estimation unit 155 may allow kinetic information being acquired to undergo machine learning to estimate a hand gesture.

The display controller 147 causes the hand gesture being estimated and a character and an indicator corresponding to the hand gesture to appear (step S150).

FIG. 13 is an explanatory diagram schematically illustrating the field of view VR of the user after execution of step S150. In FIG. 13, the external scenery SC is omitted as with FIG. 12. As illustrated in FIG. 13, the display region PN displays a hand gesture display image HS1, a character display image KC1, an image (hereinafter referred to as “information image”) Inf representing information to the user, and an image (hereinafter referred to as “indicator image”) IG representing an indicator.

The hand gesture display image HS1 is a display image representing the hand gesture estimated in step S140 described above. In the example illustrated in FIG. 13, the hand gesture display image HS1 illustrates a model image representing the hand gesture (a movement of a hand, a thumb, and fingers used for sign language) corresponding to the character “i”. The hand gesture is not a captured image of the hand of the user, but is an image prepared beforehand. The character display image KC1 is a display image representing a character corresponding to the hand gesture display image HS1. In the example illustrated in FIG. 13, the character display image KC1 illustrates “i” in text. A character corresponding to a hand gesture can be identified by referring to the character data storage unit 126 for the kinds of hand gestures. In step S140, by causing the hand gesture display image HS1 and the character display image KC1 to appear the user can recognize whether a hand gesture estimated from kinetic information corresponds to a hand gesture actually performed by the user, as well as whether a character corresponds to a character that the user has desired to enter.

The information image Inf is a display image representing information prompting the user to take further action. In the example illustrated in FIG. 13, the information image Inf displays the text “The character you have entered is here. “OK” to confirm.”, prompting the user to enter “OK”.

The indicator image IG displays a ratio of a total value of myoelectric potentials detected by the myoelectric sensors MS1 to MS12. In the example illustrated in FIG. 13, the indicator image IG has a double circle shape. The inside circle displays a text character, i.e., 20%, whereas the outside circle has a hatched area displaying 20%. By displaying the indicator image IG, the user can easily recognize how much a myoelectric potential is further required in average for the thumbs and fingers LF1 to LF5 and RF1 to RF5 in order to cause a desired hand gesture to be accepted. The indicator image IG may display an average value of myoelectric potentials detected by the myoelectric sensors MS1 to MS12 or a maximum myoelectric potential among myoelectric potentials detected by the myoelectric sensors MS1 to MS12.

As illustrated in FIG. 9, the kinetic information acquisition unit 153 acquires kinetic information (step S160). Step S160 is similar to step S100 and step S130 described above, and its detailed description is omitted. The hand gesture estimation unit 155 estimates a hand gesture from the kinetic information being acquired and determines whether the hand gesture being estimated corresponds to the hand gesture indicative of “determination of input signal” (step S170).

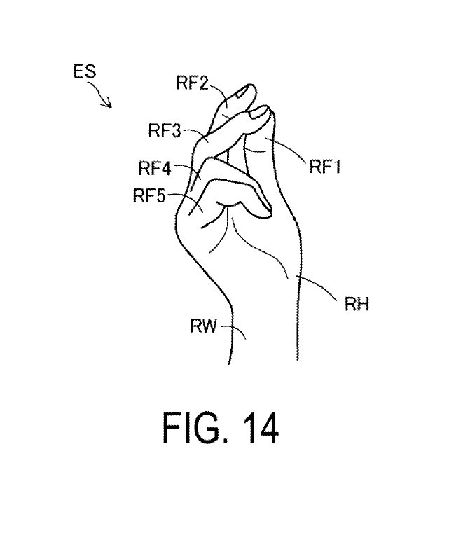

FIG. 14 is an explanatory diagram schematically illustrating a hand gesture ES indicative of “determination of input signal”. In FIG. 14, similar to FIG. 10, illustration of the myoelectric sensors MS1 to MS12 illustrated in FIG. 8 is omitted. As illustrated in FIG. 14, in the exemplary embodiment, the hand gesture ES indicative of “determination of input signal” denotes that the thumb RF1 and the third finger RF3 of the right hand RH of the user of the HMD 100 make a sound (“finger snap”). In the exemplary embodiment, why the hand gesture ES indicative of “determination of input signal” is specified to a movement of making a sound with the thumb RF1 and the third finger RF3 of the right hand RH is that such a movement of making a sound with a thumb and a finger is not specified in movements of hands, thumbs, and/or fingers for sign language, and therefore a hand gesture of entering a character using a sign language and the hand gesture of performing “determination of input signal” can be clearly distinguished from each other.

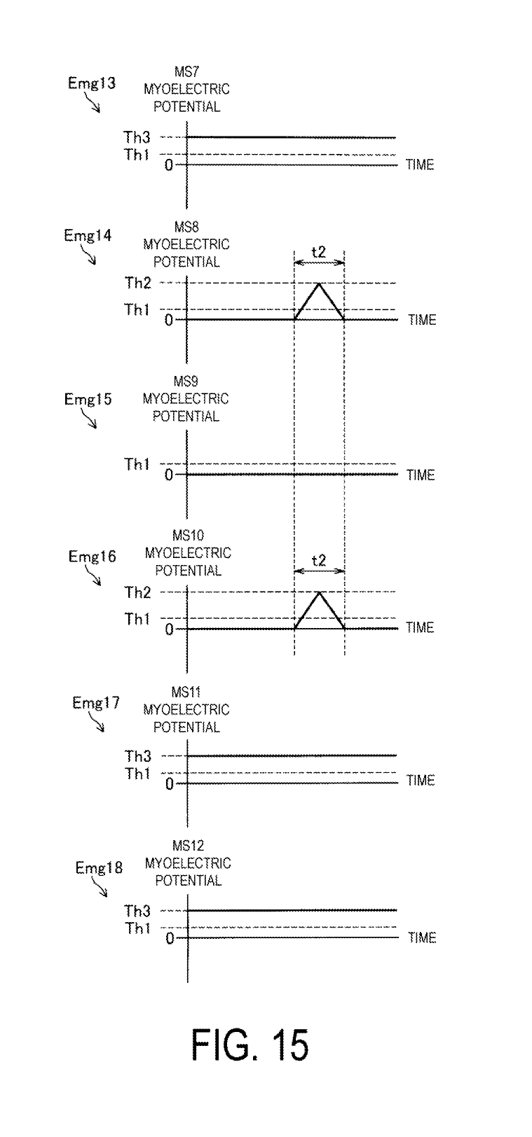

FIG. 15 is an explanatory diagram schematically illustrating temporal changes in myoelectric potential when the hand gesture ES indicative of “determination of input signal” is performed. FIG. 15 illustrates myoelectric waveforms Emg13 to Emg18 acquired from results of detection by the myoelectric sensor MS7 to MS12 for the predetermined time. In the exemplary embodiment, the hand gesture indicative of “determination of input signal” is performed with the right hand RH. Therefore, results of detection by the myoelectric sensors MS1 to MS6 attached to the left hand LH are not required. Therefore, in FIG. 15, illustration of myoelectric waveforms acquired from results of detection by the myoelectric sensors MS1 to MS6 attached to the left hand LH is omitted.

The top in FIG. 15 illustrates the myoelectric waveform Emg13 of the seventh myoelectric sensor MS7 attached to the right arm RW of the right hand RH. The second row from the top illustrates the myoelectric waveform Emg14 of the eighth myoelectric sensor MS8 attached to the thumb RF1 of the right hand RH. The third row from the top illustrates the myoelectric waveform Emg15 of the ninth myoelectric sensor MS9 attached to the index finger RF2 of the right hand RH. The fourth row from the top illustrates the myoelectric waveform Emg16 of the tenth myoelectric sensor MS10 attached to the third finger RF3 of the right hand RH. The fifth row from the top illustrates the myoelectric waveform Emg17 of the eleventh myoelectric sensor MS11 attached to the fourth finger RF4 of the right hand RH. The sixth row from the top illustrates the myoelectric waveform Emg18 of the twelfth myoelectric sensor MS12 attached to the fifth finger RF5 of the right hand RH.

In the myoelectric waveform Emg13 to Emg17, vertical axes indicate the myoelectric potentials detected by the myoelectric sensor MS7 to MS12, whereas horizontal axes indicate time. As described above, the hand gesture ES indicative of “determination of input signal” is making a sound with the thumb RF1 and the third finger RF3 of the right hand RH. Therefore, as illustrated in the myoelectric waveform Emg14, the myoelectric potential of the thumb RF1 gradually increases from 0 millivolt [mV] during a time t2, reaches a second threshold value Th2 greater than the first threshold value Th1, and then decreases to 0 millivolt [mV]. At a time when the myoelectric potential has reached the second threshold value Th2, the thumb RF1 comes into contact with the third finger RF3 to make a sound. Since the third finger RF3 is moved similar to the thumb RF1, the myoelectric waveform Emg16 of the third finger RF3 has a shape similar to the shape of the myoelectric waveform Emg13 of the thumb RF1.

The time t2 described above is 10 milliseconds, for example. The time t2 may be another desired time than 10 milliseconds. The second threshold value Th2 described above is 20 millivolts [mV], for example. The second threshold value Th2 may be set to a desired value greater than the first threshold value Th1, other than 20 millivolts [mV].

As illustrated in FIG. 14, the index finger RF2 is almost fully stretched in the hand gesture ES indicative of “determination of input signal”. Therefore, the myoelectric potential is stationary at 0 millivolt [mV], as illustrated in the myoelectric waveform Emg15 in FIG. 15. As illustrated in FIG. 14, the fourth finger RF4 and the fifth finger RF5 are folded in the hand gesture ES indicative of “determination of input signal”. Therefore, the myoelectric potentials are each stationary at a third threshold value Th3, as illustrated in the myoelectric waveforms Emg17 and Emg18 in FIG. 15. The third threshold value Th3 is 10 millivolts [mV], for example. The third threshold value Th3 may be set to a desired value less than the second threshold value Th2.

As illustrated in FIG. 14, the right arm RW is raised in the hand gesture ES indicative of “determination of input signal”. Therefore, the myoelectric potential is stationary at the third threshold value Th3, as illustrated in the myoelectric waveform Emg13 in FIG. 15. The myoelectric potentials of the fourth finger RF4, the fifth finger RF5, and the right arm RW may not be each constant to the third threshold value Th3, but may be each constant to another myoelectric potential than the third threshold value Th3.

As illustrated in FIG. 9, when it is determined that the hand gesture being estimated does not correspond to the hand gesture ES indicative of “determination of input signal” (step S170: NO), step S130 described above is then executed. On the other hand, when it is determined that the hand gesture being estimated corresponds to the hand gesture ES indicative of “determination of input signal” (step S170: YES), the input signal generating unit 157 generates and outputs an input signal corresponding to the hand gesture being estimated (step S180). Specifically, the input signal generating unit 157 generates an input signal about a character corresponding to a hand gesture estimated already before the hand gesture ES indicative of “determination of input signal” is performed. The input signal being generated is output to the input and output controller 151.

The kinetic information acquisition unit 153 determines whether end of character entry should be invoked (step S190). Specifically, after the hand gesture ES indicative of “determination of input signal” is performed, the kinetic information acquisition unit 153 measures a time of how long no kinetic information is acquired. When the time does not exceed a predetermined time, the kinetic information acquisition unit 153 determines that end of character entry should not be invoked (step S190: NO). Step S130 described above is then executed. On the other hand, when the measured time exceeds the predetermined time, the kinetic information acquisition unit 153 determines that end of character entry should be invoked (step S190: YES). The character input processing thus ends.

With the HMD 100 according to the exemplary embodiment described above, kinetic information about muscle movements of hands and arms of a user of the HMD 100 is acquired, a hand gesture specified beforehand is estimated based on the muscle movements indicated in the kinetic information being acquired, and an input signal specified beforehand is generated in accordance with the hand gesture being estimated. Therefore, entries can be easily and precisely made in the HMD 100. In addition, a potential at a skin surface when a muscle movement occurs is acquired, the myoelectric waveforms Emg1 to Emg18 indicative of temporal changes in the potential being acquired are used, and a hand gesture is estimated. Therefore, the hand gesture can be precisely estimated.

Since the hand gesture display image HS1 representing a hand gesture being estimated and the character display image KC1 representing a character corresponding to the hand gesture are displayed, the hand gesture being estimated and the character corresponding to the hand gesture can be notified to the user. In addition, when a hand gesture being estimated corresponds to the hand gesture ES indicative of determination of input signal corresponding to a hand gesture estimated already, the input signal is output. Therefore, the input signal can be precisely output, and also erroneous entry can be suppressed.

### B. Other Exemplary Embodiments

### B1. Other Exemplary Embodiment 1

In the exemplary embodiment described above, the display controller 147 causes both the hand gesture display image HS1 and the character display image KC1 to appear. However, the present disclosure is not limited to this configuration. For example, the display controller 147 may cause only the hand gesture display image HS1 or the character display image KC1 to appear. That is, in general, the display controller 147 may cause at least either of the hand gesture display image HS1 or the character display image KC1 to appear. For example, the display controller 147 may not cause the hand gesture display image HS1 and the character display image KC1 to appear. Such a configuration also produces effects similar to the effects of the exemplary embodiment described above.

### B2. Other Exemplary Embodiment 2

In the exemplary embodiment described above, the input signal generating unit 157 outputs an input signal when a hand gesture being estimated corresponds to the hand gesture ES indicative of “determination of input signal”. However, the present disclosure is not limited to this configuration. For example, the input signal generating unit 157 may not output an input signal when a hand gesture being estimated corresponds to the hand gesture ES indicative of “determination of input signal”, or may output an input signal when a hand gesture does not correspond to the hand gesture ES indicative of “determination of input signal”. Such a configuration also produces effects similar to the effects of the exemplary embodiments described above.

### B3. Other Exemplary Embodiment 3

In the exemplary embodiments described above, the hand gesture ES indicative of “determination of input signal” is making a sound with the thumb RF1 and the third finger RF3 of the right hand RH. However, the present disclosure is not limited to this configuration. For example, the hand gesture ES indicative of “determination of input signal” may be clapping with the left hand LH and the right hand RH. In the configuration, when one of detection values of the myoelectric sensors MS1 to MS12 exceeds the second threshold value Th2 during the time t2 only, the hand gesture estimation unit 155 may estimate that the left hand LH and the right hand RH are clapped. For example, the hand gesture ES indicative of “determination of input signal” may be making a sound at a joint of one of the thumb and the fingers of the right hand RH with the left hand LH. That is, in general, the hand gesture ES indicative of “determination of input signal” may be another desired hand gesture, as long as the left hand LH and the right hand RH are used to make a sound. Such a configuration also produces effects similar to the effects of the exemplary embodiments described above.

### B4. Other Exemplary Embodiment 4

In the exemplary embodiments and other Exemplary Embodiment 3 described above, the input signal generating unit 157 may generate and output, when one of body movements specified beforehand as indicative of “determination of input signal” is detected, an input signal corresponding to a hand gesture being estimated. The “body movements specified beforehand” correspond to movements including “stepping”, “clicking one's tongue”, and “making a sound with teeth”, for example. The kinetic information acquisition unit 153 may detect “stepping” from results of detection by myoelectric sensors MS attached to the legs of the user of the HMD 100. The kinetic information acquisition unit 153 may detect “clicking one's tongue” and “making a sound with teeth” from results of detection by myoelectric sensors MS attached to the head of the user of the HMD 100. The input signal generating unit 157 may generate and output an input signal when the hand gesture ES indicative of “determination of input signal” is performed and one of the body movements specified beforehand described above is detected. Such a configuration also produces effects similar to the effects of the exemplary embodiments and other Exemplary Embodiment 3 described above.

### B5. Other Exemplary Embodiment 5

In the exemplary embodiments described above, the hand gesture estimation unit 155 may use, in addition to kinetic information, an image captured by the camera 61 to estimate a hand gesture. In the configuration, the image being captured may be analyzed, the shapes of the left hand LH and the right hand RH may be identified, pattern matching may be performed between the identified shapes of the hands and the kinds of hand gestures stored in the hand gesture data storage unit 125, and a hand gesture may be estimated. The display controller 147 may cause the image display unit 20 to display a display image representing the identified shapes of the hands, in addition to the hand gesture display image HS1 and the character display image KC1. Such a configuration also produces effects similar to the effects of the exemplary embodiments described above.

### B6. Other Exemplary Embodiment 6

In the exemplary embodiments described above, the input signal generating unit 157 generates an input signal about a character corresponding to a hand gesture. However, the present disclosure is not limited to this configuration. For example, the input signal generating unit 157 may generate an input signal about a command. In the configuration, a map of the kinds of hand gestures and commands associated with each other may be stored beforehand in the hand gesture data storage unit 125, and an input signal about a command specified in accordance with a hand gesture being estimated may be generated. For example, the input signal generating unit 157 may generate an input signal about one of instructions such as page feeding, page returning, zoom out, and zoom in a Web page, for example. That is, in general, effects similar to the effects of the exemplary embodiments are produced, as long as a configuration generates an input signal specified beforehand in accordance with a hand gesture being estimated.

### B7. Other Exemplary Embodiment 7

In the exemplary embodiments described above, the kinds of hand gestures and the kinetic information are associated with each other beforehand. However, in addition to this, the kinds of hand gestures and the kinetic information may be associated with each other per user. For example, when a user is left-handed, the kinds of hand gestures may be associated with hand gestures for the left hand. For example, for a female user, the kinetic information such as the threshold values Th1 to Th3 for myoelectric potentials may be associated with lesser values than values to be applied for a male user. Such a configuration also produces effects similar to the effects of the exemplary embodiments described above.

### B8. Other Exemplary Embodiment 8

In the exemplary embodiments described above, the kinetic information acquisition unit 153 acquires kinetic information from the myoelectric sensors MS1 to MS12. However, the present disclosure is not limited to this configuration. For example, in a configuration where gyro sensors are attached to arms and hands of a user, the kinetic information acquisition unit 153 may acquire, from the gyro sensors, movements of the hands and the arms of the user of the HMD 100. For example, the kinetic information acquisition unit 153 may acquire a sound collected by the microphone 63. In the configuration, the hand gesture estimation unit 155 may use the sound entry being acquired to estimate a hand gesture. In a configuration where the left hand LH and the right hand RH make a sound as the hand gesture indicative of “determination of input signal”, a sound entry can be used to precisely estimate the hand gesture indicative of “determination of input signal”. Such a configuration also produces effects similar to the effects of the exemplary embodiments described above.

### B9. Other Exemplary Embodiment 9

In the exemplary embodiments described above, the myoelectric sensors MS1 to MS12 are myoelectric sensors including surface electrodes. However, the myoelectric sensors may include needle electrodes or wire electrodes, instead of surface electrodes. For example, a desired number of the myoelectric sensors MS may be attached to the arms LW and RW, the thumbs and fingers RF1 to RF5 and LF1 to LF5. The myoelectric sensors MS1 and MS7 attached to the arms LW and RW are arm ring type myoelectric sensors. However, wrist watch type myoelectric sensors may be used, instead of arm ring type myoelectric sensors. For example, instead of the myoelectric sensors, such muscle activity detection devices may be used that are capable of detecting potentials generated when cells and tissues, such as muscles and nerves, are excited. The muscle activity detection devices may be pulse measuring devices, for example. When pulse measuring devices are adopted, such a characteristic that hemoglobin in blood absorbs light may be used to measure pulses to acquire the measured pulses as a muscle activity. Specifically, sensors included in the pulse measuring devices attached to arms of a user of the HMD 100 may be allowed to irradiate light on blood vessels in skin, photoreceptor elements may be allowed to measure light that has not been absorbed by hemoglobin, but returned from inside of the skin, and pulses may be measured based on an amount of the light received. Such a configuration also produces effects similar to the effects of the exemplary embodiments described above.

### B10. Other Exemplary Embodiment 10

In the exemplary embodiments described above, the HMD 100 and the myoelectric sensors MS1 to MS12 are coupled based on wireless coupling. However, wired coupling may be used, instead of the wireless coupling. Such a configuration also produces effects similar to the effects of the exemplary embodiments described above.

### B11. Other Exemplary Embodiment 11

In the exemplary embodiments described above, the hand gesture ES indicative of “start of character entry” corresponds to a state when the left hand LH and the right hand RH are closed. However, the present disclosure is not limited to this configuration. For example, the hand gesture ES indicative of “start of character entry” may be identical to the hand gesture SS indicative of “determination of input signal”. For example, the hand gesture ES indicative of “start of character entry” may correspond to a state when the left hand LH is open (the state is referred to as “paa (open palm shape)” in Japan), whereas the right hand RH is closed (the state is referred to as “guu (fist shape)” in Japan). Such a configuration also produces effects similar to the effects of the exemplary embodiments described above.

### B12. Other Exemplary Embodiment 12

In the exemplary embodiments described above, the OLED units 221 and 241 are configured to include the OLED panels 223 and 243 and the OLED drive circuits 225 and 245 that respectively drive the OLED panels 223 and 243, and the OLED panels 223 and 243 are each a self-light-emitting display panel including light emitting elements that emit light by organic electro-luminescence. However, the present disclosure is not limited to this configuration. Furthermore, each of the OLED panels 223 and 243 includes a plurality of pixels arranged in a matrix, and each of the plurality of pixels corresponds to a unit including one element of R, one element of G, and one element of B. However, the present disclosure is not limited to this configuration. For example, the right display unit 22 and the left display unit 24 each may be configured as a video element that includes an OLED panel serving as a light source unit and a modulation element to modulate light emitted by the light source unit to output image light including a plurality of colors of light. Note that the modulation device for modulating the light emitted by the OLED panel is not limited to a configuration in which a transmissive liquid crystal panel is adopted. For example, a reflective liquid crystal panel may be used instead of the transmissive liquid crystal panel, or a digital micro-mirror device or a laser scan type laser retinal projection HMD may be used. Such a configuration also produces effects similar to the effects of the exemplary embodiments described above.

### B13. Other Exemplary Embodiment 13

In the exemplary embodiments described above, the control function unit 150 is included in the control device 10. Instead of the configuration, however, in a configuration where the image display unit 20 includes a main processor, the control function unit 150 may be included in the image display unit 20. Such a configuration also produces effects similar to the effects of the exemplary embodiments described above.

### B14. Other Exemplary Embodiment 14

In the exemplary embodiments described above, the muscle activity detection devices detect muscle movements of the hands and arms. However, in addition to the movements, movements of other parts of the body of the user of the HMD 100 may be detected. Specifically, muscle movements of feet may be detected with muscle activity detection devices attached to the feet thumbs and fingers, feet bottoms, and thighs, for example. For example, muscle movements of the chin may be detected with a muscle activity detection device attached to the head. For example, muscle movements of a whole body may be detected with muscle activity detection devices applied and attached to the body at predetermined intervals. That is, in general, such a configuration that muscle activity detection devices detect muscle movements of a body of a user of the HMD 100 also produces effects similar to the effects of the exemplary embodiments described above.

### B15. Other Exemplary Embodiment 15

In the exemplary embodiments described above, the indicator image IG displays a ratio of a total value of myoelectric potentials detected by the myoelectric sensors MS1 to MS12. However, the present disclosure is not limited to this configuration. For example, when a myoelectric potential required for determining the hand gesture ES indicative of “determination of input signal” in step S170 described above is regarded as 100%, a ratio of a total value of myoelectric potentials detected by the myoelectric sensors MS1 to MS12 may be displayed. For example, in accordance with the ratio, the indicator image IG may be displayed in a different color, the indicator image IG may be flash, or the indicator image IG may be displayed in a different shape. This allows a user having performed a movement (gesture) below a degree attaining determination of input signal to easily know how much a further movement is required to allow the input signal to be determined. For example, after step S180 described above is executed, the display controller 147 may display the indicator image IG in a display aspect different in color, for example, from a display aspect before step S180 is executed. This can notify, to a user of the HMD 100, that an input signal is generated and output. Such a configuration also produces effects similar to the effects of the exemplary embodiments described above.

### B16. Other Exemplary Embodiment 16

In other Exemplary Embodiment 15 described above, a set value for a myoelectric potential required for determining the hand gesture ES indicative of “determination of input signal” may vary depending on a user. For example, for a female user, a lesser value may be set than a value for a male user. For example, such a configuration may be adopted that a set value can be changed in accordance with how easy a user can perform an operation. The set value may be associated with a user and stored in the hand gesture data storage unit 125. Such a configuration also produces effects similar to the effects of other Exemplary Embodiment 15 described above.

The present disclosure is not limited to the exemplary embodiments described above. Rather, the present disclosure can be achieved in various configurations, to an extent that such configurations fall within the scope of the present disclosure. For example, technical features of the exemplary embodiments, which correspond to the technical features of the exemplary embodiments described in the summary of the present disclosure, may be appropriately replaced or combined to address some or all of the above-identified problems or to achieve some or all of the above-described advantages. Any of the technical features may be deleted as appropriate unless the technical feature is described in the specification as indispensable.

### C. Other Exemplary Embodiments

(1) An exemplary embodiment of the present disclosure provides a transmissive head-mounted display apparatus. The transmissive head-mounted display apparatus includes an image display unit configured to transmit an external scenery and display an image of a display target viewable with the external scenery, a kinetic information acquisition unit configured to acquire information about the muscle movement, detected by a muscle activity detection device configured to detect a muscle movement of a body of a user of the transmissive head-mounted display apparatus, a hand gesture estimation unit configured to estimate a hand gesture specified beforehand, based on the muscle movement indicated in the information that has been acquired, and an input signal generation unit configured to generate an input signal specified beforehand in accordance with the hand gesture that is estimated.

With the transmissive head-mounted display apparatus according to the exemplary embodiment, information about muscle movements of a body of a user of the transmissive head-mounted display apparatus is acquired, a hand gesture specified beforehand is estimated based on the muscle movements indicated by the information that has been acquired, and an input signal specified beforehand is generated in accordance with the hand gesture that is estimated. Therefore, entries can be easily and precisely made in the transmissive head-mounted display apparatus.

(2) In the transmissive head-mounted display apparatus according to the exemplary embodiment described above, the kinetic information acquisition unit may acquire, as the information, a potential at a skin surface when the muscle movement occurs, and the hand gesture estimation unit may use a myoelectric waveform indicative of a temporal change in the potential being acquired in order to estimate the hand gesture. With the transmissive head-mounted display apparatus according to the exemplary embodiment, a potential at a skin surface when a muscle movement occurs is acquired, myoelectric waveforms indicative of temporal changes in the potential being acquired are used, and a hand gesture is estimated. Therefore, the hand gesture can be precisely estimated.

(3) The transmissive head-mounted display apparatus according to the exemplary embodiment described above may further include a display controller configured to cause at least one of a display image representing the hand gesture that is estimated and a display image representing a character corresponding to the hand gesture, to be displayed. With the transmissive head-mounted display apparatus according to the exemplary embodiment, at least one of a display image representing a hand gesture that is estimated and a display image representing a character corresponding to the hand gesture is displayed. Therefore, the hand gesture that is estimated or the character corresponding to the hand gesture can be notified to the user.

(4) In the transmissive head-mounted display apparatus according to the exemplary embodiment described above, the input signal generating unit may output the input signal when the hand gesture that is estimated corresponds to the hand gesture indicative of determination of the input signal corresponding to a hand gesture that has been already estimated. With the transmissive head-mounted display apparatus according to the exemplary embodiment, when a hand gesture that is estimated corresponds to the hand gesture indicative of determination of input signal corresponding to hand gesture that has been already estimated, the input signal is output. Therefore, the input signal can be precisely output, and also erroneous entry can be suppressed.

(5) In the transmissive head-mounted display apparatus according to the exemplary embodiment described above, the hand gesture indicative of determination of the input signal may be a movement of making a sound. With the transmissive head-mounted display apparatus according to the exemplary embodiment, the hand gesture indicative of determination of input signal is a movement of making a sound. When a hand gesture corresponding to a movement of hands, thumbs, and/or fingers for sign language is used as a hand gesture used for entering a character, for example, since a movement of making a sound is not specified in movements of hands, thumbs, and/or fingers for sign language, a hand gesture used for entering a character, for example and a hand gesture indicative of determination of input signal can be clearly distinguished from each other, and also erroneous entry can be suppressed.

(6) In the transmissive head-mounted display apparatus according to the exemplary embodiment described above, the input signal generating unit may output the input signal when a movement of the body is detected, the movement having been specified beforehand as indicative of determination of the input signal. With the transmissive head-mounted display apparatus according to the exemplary embodiment, when the body movement specified beforehand is detected, an input signal is output. Therefore, determination of the input signal can be precisely performed.

(7) In the transmissive head-mounted display apparatus according to the exemplary embodiment described above, a display controller may be further included, and the kinetic information acquisition unit may acquire, as the information, a potential at a skin surface when the muscle movement occurs, and the display controller may cause an indicator image indicative of the potential being acquired to be displayed. With the transmissive head-mounted display apparatus according to the exemplary embodiment, an indicator image indicative of a potential being acquired is displayed. Therefore, a user can easily recognize how much (amount) the user has performed a muscle movement.

(8) In the transmissive head-mounted display apparatus according to the exemplary embodiment described above, the display controller may cause, when the hand gesture that is estimated corresponds to the hand gesture indicative of determination of the input signal corresponding to a hand gesture that has been already estimated, a display aspect of the indicator image to be different from a display aspect of the indicator image when the hand gesture that is estimated does not correspond to the hand gesture indicative of determination of the input signal. With the transmissive head-mounted display apparatus according to the exemplary embodiment, when a hand gesture that is estimated corresponds to a hand gesture indicative of determination of input signal corresponding to hand gesture that has been already estimated, a display aspect of an indicator image is displayed differently from a display aspect of an indicator image when a hand gesture that is estimated is not a hand gesture indicative of determination of input signal. Therefore, a user can easily recognize that the gesture performed by the user is estimated as a gesture indicative of determination of input signal.

(9) In the transmissive head-mounted display apparatus according to the exemplary embodiment described above, an imaging unit may be further included, and the hand gesture estimation unit may estimate the hand gesture, based on a shape of a hand of the user in an image captured through imaging. With the transmissive head-mounted display apparatus according to the exemplary embodiment, the imaging unit is included, and a hand gesture is estimated based on a shape of a hand of a user in an image captured through imaging. Therefore, the hand gesture can be precisely estimated, compared with a configuration where no captured image is used for estimating a hand gesture.

The present disclosure may be achieved in various exemplary embodiments. For example, the exemplary embodiments of the present disclosure may include a display control method for a transmissive head-mounted display apparatus, a computer program for implementing the display control method, and a recording medium in which the computer program is recorded.

What is claimed is:

1. A transmissive head-mounted display apparatus comprising:
an image display unit configured to transmit an external scenery and display an image of a display target viewable with the external scenery;
a kinetic information acquisition unit configured to acquire information about a muscle movement detected by a muscle activity detection device configured to detect a muscle movement of a body of a user of the transmissive head-mounted display apparatus;
a hand gesture estimation unit configured to estimate a hand gesture specified beforehand, based on the muscle movement indicated in the information that has been acquired; and
an input signal generation unit configured to generate an input signal specified beforehand in accordance with the hand gesture that is estimated.

2. The transmissive head-mounted display apparatus according to claim 1, wherein
the kinetic information acquisition unit acquires, as the information, a potential at a skin surface when the muscle movement occurs, and
the hand gesture estimation unit uses a myoelectric waveform indicative of a temporal change in the potential being acquired in order to estimate the hand gesture.

3. The transmissive head-mounted display apparatus according to claim 1, further comprising a display controller configured to cause at least one of a display image representing the hand gesture that is estimated and a display image representing a character corresponding to the hand gesture, to be displayed.

4. The transmissive head-mounted display apparatus according to claim 1, wherein the input signal generation unit outputs the input signal when the hand gesture that is estimated corresponds to the hand gesture indicative of determination of the input signal corresponding to a hand gesture that has been already estimated.

5. The transmissive head-mounted display apparatus according to claim 4, wherein the hand gesture indicative of determination of the input signal is a movement of making a sound.

6. The transmissive head-mounted display apparatus according to claim 1, wherein the input signal generation unit outputs the input signal when a movement of the body is detected, the movement having been specified beforehand as indicative of determination of the input signal.

7. The transmissive head-mounted display apparatus according to claim 2, further comprising a display controller, wherein
the kinetic information acquisition unit acquires, as the information, a potential at a skin surface when the muscle movement occurs, and
the display controller causes an indicator image indicative of the potential being acquired to be displayed.

8. The transmissive head-mounted display apparatus according to claim 7, wherein the display controller causes, when the hand gesture that is estimated corresponds to the hand gesture indicative of determination of the input signal corresponding to a hand gesture that has been already estimated, a display aspect of the indicator image to be different from a display aspect of the indicator image when the hand gesture that is estimated does not correspond to the hand gesture indicative of determination of the input signal.

9. The transmissive head-mounted display apparatus according to claim 1, further comprising an imaging unit, wherein
the hand gesture estimation unit estimates the hand gesture, based on a shape of a hand of the user in an image captured through imaging.

10. A display control method for a transmissive head-mounted display apparatus including an image display unit configured to transmit an external scenery and display an image of a display target viewable with the external scenery, the display control method comprising:
acquiring information about a muscle movement detected by a muscle activity detection device configured to detect a muscle movement of a body of a user of the transmissive head-mounted display apparatus;
estimating a hand gesture specified beforehand, based on the muscle movement indicated in the information that has been acquired; and
generating an input signal specified beforehand in accordance with the hand gesture that is estimated.

11. A non-transitory computer-readable storage medium storing a computer program, the computer program for implementing a display control in a transmissive head-mounted display apparatus including an image display unit configured to transmit an external scenery and display an image of a display target viewable with the external scenery, the computer program causing a computer to implement functions comprising:
acquiring information about a muscle movement, detected by a muscle activity detection device configured to detect a muscle movement of a body of a user of the transmissive head-mounted display apparatus;
estimating a hand gesture specified beforehand, based on the muscle movement indicated in the information that has been acquired; and
generating an input signal specified beforehand in accordance with the hand gesture that is estimated.
